(19) United States
(12) Patent Application Publication
(10) Pub. No.: US 2010/0168099 A1
(43) Pub. Date: Jul. 1, 2010

(54) 1H-QUINOLIN-4-ONE COMPOUNDS, WITH AFFINITY FOR THE GABA RECEPTOR, PROCESSES, USES AND COMPOSITIONS

(75) Inventors: José Luis Falcö, Barcelona (ES); Albert Palomer, Barcelona (ES); Antonio Guglietta, Molins De Rei (ES)

Correspondence Address: BIRCH STEWART KOLASCH & BIRCH, PO BOX 747, FALLS CHURCH, VA, 22040-0747, US

(73) Assignee: FERRER INTERNACIONAL, S.A., Barcelona (ES)

(21) Appl. No.: 12/377,103

(22) PCT Filed: Aug. 9, 2007
(86) PCT No.: PCT/EP2007/058288

§ 371 Date: Jun. 9, 2009

Related U.S. Application Data
(60) Provisional application No. 60/836,666, filed on Aug. 10, 2006.

(30) Foreign Application Priority Data
Aug. 10, 2006 (EP) 06118720.9

(51) Int. Cl. A61K 31/5377; C07D 215/02; C07D 413/02; C07D 401/02; A61K 31/47; A61K 31/497; A61P 25/08; A61P 25/20; A61P 21/02

(52) U.S. Cl. 514/235.2; 546/153; 544/128; 544/363; 514/312; 514/253.07

(57) ABSTRACT

The invention provides new 1H-quinolin-4-one compounds of formula (I), wherein R₁, R₂, R₃, R₄ and R₅ have different meanings, and pharmaceutically acceptable salts and hydrates thereof. Compounds of formula (I) are useful for treating or preventing diseases associated with GABA_{A} receptors modulation, anxiety, epilepsy, sleep disorders including insomnia, and for inducing sedation-hypnosis, anesthesia, sleep and muscle relaxation. The invention also provides synthetic procedures for preparing said compounds.

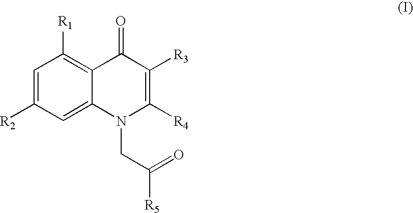

### TECHNICAL FIELD

This invention is directed to a new class of agents with affinity for GABA_{A} receptor. The invention concerns specifically new 1H-quinolin-4-one compounds which are useful for treating or preventing anxiety, epilepsy and sleep disorders including insomnia, and for inducing sedation-hypnosis, anaesthesia, sleep and muscle relaxation.

### BACKGROUND OF THE INVENTION

GABA_{A} receptor (γ-aminobutyric acid_{A}) is a pentameric protein which forms a membrane ion channel. GABA_{A} receptor is implicated in the regulation of sedation, anxiety, muscle tone, epileptogenic activity and memory functions. These actions are due to defined subunits of GABA_{A} receptor, particularly the α₁- and α₂-subunits.

Sedation is modulated by the α₁-subunit. Zolpidem is characterized by a high affinity for the α₁-receptors and its sedative and hypnotic action is mediated by these receptors in vivo. Similarly, the hypnotic action of zaleplon is also mediated by the α₁-receptors.

The anxiolytic action of diazepam is mediated by the enhancement of GABAergic transmission in a population of neurons expressing the α₂-receptors. This indicates that the α₂-receptors are highly specific targets for the treatment of anxiety.

Muscle relaxation in diazepam is mainly mediated by α₂-receptors, since these receptors exhibit a highly specific expression in spinal cord.

The anticonvulsant effect of diazepam is partly due to α₁-receptors. In diazepam, a memory-impairing compound, anterograde amnesia is mediated by α₁-receptors.

GABA_{A} receptor and α₁- and α₂-subunits have been widely reviewed by H. Möhler et al. (J. Pharmacol. Exp. Ther., 300, 2-8, 2002); H. Möhler et al. (Curr. Opin. Pharmacol., 1, 22-25, 2001); U. Rudolph et al. (Nature, 401, 796-800, 1999); and D. J. Nutt et al. (Br. J. Psychiatry, 179, 390-396, 2001).

Diazepam and other classical benzodiazepines are extensively used as anxiolytic agents, hypnotic agents, anticonvulsants and muscle relaxants. Their side effects include anterograde amnesia, decrease in motor activity and potentiation of ethanol effects.

In this context, the compounds of this invention are ligands of α₁- and α₂-GABA_{A} receptor for their clinical application in sleep disorders, preferably insomnia, anxiety and epilepsy.

Insomnia is a highly prevalent disease. Its chronicity affects 10% of the population and 30% when transitory insomnia is computed as well. Insomnia describes the trouble in getting to sleep or staying asleep and is associated with next-day hangover effects such as weariness, lack of energy, low concentration and irritability. The social and health impact of this complaint is important and results in evident socioeconomic repercussions.

Pharmacological therapy in the management of insomnia firstly included barbiturates and chloral hydrate, but these drugs elicit numerous known adverse effects, for example, overdose toxicity, metabolic induction, and enhanced dependence and tolerance. In addition, they affect the architecture of sleep by decreasing above all the duration and the number of REM sleep stages. Later, benzodiazepines meant an important therapeutic advance because of their lower toxicity, but they still showed serious problems of dependence, muscle relaxation, amnesia and rebound insomnia following discontinuation of medication.

The latest known therapeutic approach has been the introduction of non-benzodiazepine hypnotics, such as pyrrolo[3,4-b]pyrazines (zopiclone), imidazo[1,2-a]pyridines (zolpidem) and, finally, pyrazolo[1,5-a]pyrimidines (zaleplon). Later, two new pyrazolo[1,5-a]pyrimidines, indiplon and ocinaplon, have entered into development, the latter with rather anxiolytic action. All these compounds show a rapid sleep induction and have less next-day hangover effects, lower potential for abuse and lower risk of rebound insomnia than benzodiazepines. The mechanism of action of these compounds is the alosteric activation of GABA_{A} receptor through its binding to benzodiazepine binding site (C. F. P. George, The Lancet, 358, 1623-1626, 2001). While benzodiazepines are unspecific ligands at GABA_{A} receptor binding site, zolpidem and zaleplon show a greater selectivity for α₁-subunit. Notwithstanding that, these drugs still affect the architecture of sleep and may induce dependence in long-term treatments.

Research for new active compounds in the management of insomnia answers an underlying health need, because even recently introduced hypnotics still affect the architecture of sleep and may induce dependence in long-term treatments.

It is therefore desirable to focus on the development of new hypnotic agents with a lower risk of side effects.

The present invention is directed to a new class of 1H-quinolin-4-one compounds, specifically a new chemotype consisting of phenyl-substituted N-acetyl-1H-quinolin-4-one, which is active versus GABA_{A} receptor and, particularly, versus its α₁- and α₂-subunits. No related compounds belonging to this chemotype have been disclosed to present with ligand GABA_{A} receptor or any other central nervous system activity.

Compounds containing the 1H-quinolin-4-one core have been disclosed for other purposes. Thus, EP 856255 discloses the use of some 1H-quinolin-4-ones as marine antifouling agents, WO 2001012607 discloses some other 1H-quinolin-4-ones which increase the activity of cytotoxic agents, and finally WO 2002022074 discloses the preparation of some 1H-3-phenyl-quinolin-4-ones as intimal neoproliferation inhibitors.

The compounds of the present invention have a high affinity for α₁- and α₂-GABA_{A} receptors. The in vitro activities shown by these compounds are consistent with those in vivo results obtained in sedation-hypnosis tests.

Consequently, the compounds of this invention are useful in the treatment and prevention of all those diseases mediated by GABA_{A} receptor α₁- and α₂-subunits. Non-limitative examples of such diseases are sleep disorders, preferably insomnia, anxiety and epilepsy. Non-limitative examples of the relevant indications of the compounds of this invention are all those diseases or conditions, such as insomnia or anesthesia, in which an induction of sleep, an induction of sedation or an induction of muscle relaxation are needed.

### SUMMARY OF THE INVENTION

The present invention relates to a compound of formula (I):

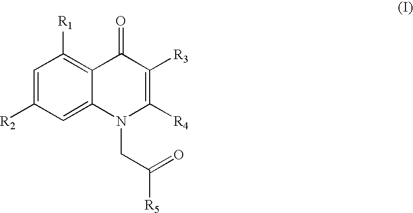

and pharmaceutically acceptable salts and hydrates thereof, which are ligands of GABA_{A} receptor.

It is another object of this invention to provide synthetic procedures for preparing the compounds of formula (I). Novel methods of treating or preventing diseases associated with GABA_{A} receptor modulation such as anxiety, epilepsy and sleep disorders including insomnia, and for inducing sedation-hypnosis, anesthesia, sleep and muscle relaxation by administering a therapeutically effective amount of said compounds are also within the scope of the invention.

### DETAILED DESCRIPTION OF THE INVENTION

The present invention relates to a compound of formula (I):

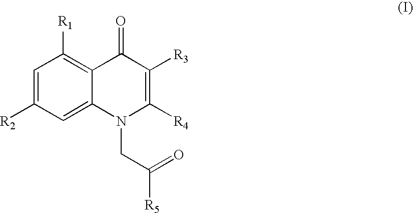

wherein R₁ and R₂ are independently selected from the group consisting of hydrogen, alkyl(C₁-C₆), Oalkyl(C₁-C₆), halogen and phenyl; R₃ and R₄ are independently selected from the group consisting of hydrogen and phenyl optionally substituted by alkyl(C₁-C₆), halogen and Oalkyl(C₁-C₆); R₅ is selected from the group consisting of NR₆R₇, N(R₈)NH₂, OH, OR₉, and a heteroaryl ring selected from the group consisting of pyridyl, furyl, thienyl, oxazolyl, isoxazolyl, imidazolyl, pyrrolyl, pyrazolyl, pyrimidinyl and pyrazinyl, wherein each heteroaryl ring contains one or two optional alkyl(C₁-C₆) substituents; R₆ and R₇ are independently selected from the group consisting of hydrogen; alkyl(C₁-C₆); alkenyl(C₂-C₆); cycloalkyl(C₃-C₆)alkyl(C₁-C₆); hydroxyalkyl(C₁-C₆); heteroaryl selected from the group consisting of pyridyl, pyrimidinyl, pyrazinyl, thiazolyl, benzothiazolyl, oxazolyl, benzoxazolyl, isoxazolyl, benzisoxazolyl, pyrazolyl, furyl, benzofuryl, thienyl, benzothienyl, thiadiazolyl, pyrrolyl, imidazolyl, benzimidazolyl, indolyl, quinolyl and isoquinolyl, wherein each heteroaryl contains one or two optional substituents independently selected from the group consisting of alkyl(C₁-C₆), Oalkyl(C₁-C₆), haloalkyl(C₁-C₆), cycloalkyl(C₃-C₆), NO₂ and COalkyl(C₁-C₆); thienylalkyl(C₁-C₆), furylalkyl(C₁-C₆), pyridylalkyl(C₁-C₆); and aryl selected from the group consisting of phenyl and indanyl, wherein each aryl contains one or two optional substituents selected from the group consisting of alkyl(C₁-C₆), haloalkyl(C₁-C₆), halogen, Ndialkyl(C₁-C₆), NHalkyl(C₁-C₆), Oalkyl(C₁-C₆), NO₂, CN, OH, NH₂, COON, COalkyl(C₁-C₆), COOalkyl(C₁-C₆), CONHalkyl(C₁-C₆), CONdialkyl(C₁-C₆) and COphenyl; or R₆ and R₇ can form, together with the nitrogen atom to which they are attached, a heterocycle selected from pyrrolidine, 3-pyrroline, 1,2,3,6-tetrahydropyridine, piperidine, morpholine, thiomorpholine and piperazine, wherein each heterocycle contains one, two, three or four optional substituents selected from the group consisting of OH, alkyl(C₁-C₆) and COalkyl(C₁-C₆); R₈ is selected from the group consisting of hydrogen and alkyl(C₁-C₆); and R₉ is alkyl(C₁-C₆); with the proviso that R₃ and R₄ cannot be hydrogen simultaneously; and pharmaceutically acceptable salts and hydrates thereof.

In a preferred embodiment R¹ is selected from the group consisting of hydrogen, alkyl(C₁-C₆), Oalkyl(C₁-C₆), halogen and phenyl; R² is selected from the group consisting of hydrogen, alkyl(C₁-C₆), halogen and phenyl; and R₃, R₄, R⁵, R₆, R₇, R₈ and R₉ are as defined above.

In another preferred embodiment R₅ is selected from the group consisting of NR₆R₇, N(R₈)NH₂ and a heteroaryl ring selected from the group consisting of pyridyl, furyl, thienyl, oxazolyl, isoxazolyl, imidazolyl, pyrrolyl, pyrazolyl, pyrimidinyl and pyrazinyl, wherein each heteroaryl ring contains one or two optional alkyl(C₁-C₆) substituents; R₆ and R₇ are as defined above, with the proviso that R₆ and R₇ are not NH₂, NH(C₁-C₄ alkyl) or N(C₁-C₄ alkyl)₂; and R₁, R₂, R₃, R₄ and R₈ are as defined above.

In another preferred embodiment, R₁ and R₂ are independently selected from hydrogen, alkyl(C₁-C₆), halogen, in particular bromine, and phenyl.

In another preferred embodiment, R₁ and R₂ are independently selected from the group consisting of hydrogen, methyl, bromine and phenyl.

In another preferred embodiment, R₃ and R₄ are independently selected from the group consisting of hydrogen, phenyl, 4-fluorophenyl, 4-chlorophenyl, p-tolyl and 4-methoxyphenyl.

In another preferred embodiment, R₅ is selected from the group consisting of OH, methoxy, ethoxy, NHNH₂, N(methyl)NH₂, 2-pyridyl, 3-pyridil, 4-pyridyl, 2-pyrazinyl, 5-methyl-2-furyl, 2-thienyl and 2,4-dimethyl-5-oxazolyl.

In another preferred embodiment, R₅ is NR₆R₇ and R₆ and R₇ are independently selected from hydrogen, alkyl(C₁-C₆); alkenyl(C₂-C₆); cycloalkyl(C₃-C₆)alkyl(C₁-C₆); hydroxyalkyl(C₁-C₆); heteroaryl selected from the group consisting of pyridyl; pyrimidinyl, optionally substituted with one or two substituents independently selected from Oalkyl(C₁-C₆) and OH; pyrazinyl; thiazolyl, optionally substituted with one or two substituents independently selected from alkyl(C₁-C₆) and NO₂; benzothiazolyl; oxazolyl, optionally substituted with one or two alkyl(C₁-C₆); benzoxazolyl; isoxazolyl, optionally substituted with one or two alkyl(C₁-C₆); benzisoxazolyl; pyrazolyl, optionally substituted with one or two substituents independently selected from alkyl(C₁-C₆) and cycloalkyl(C₁-C₆); furyl, optionally substituted with one or two alkyl(C₁-C₆); benzofuryl; thienyl, optionally substituted with one or two COalkyl(C₁-C₆); benzothienyl; thiadiazolyl, optionally substituted with one or two haloalkyl(C₁-C₆); pyrrolyl, optionally substituted with one or two alkyl(C₁-C₆); imidazolyl; benzimidazolyl; indolyl, optionally substituted with one or two alkyl(C₁-C₆); quinolyl; and isoquinolyl; thienylalkyl(C₁-C₆), furylalkyl(C₁-C₆), pyridylalkyl(C₁-C₆); phenyl, optionally substituted with one or two substituents independently selected from alkyl(C₁-C₆), haloalkyl(C₁-C₆), Oalkyl(C₁-C₆), halogen, NHalkyl(C₁-C₆), and Ndialkyl(C₁-C₆); and indanyl; or R₆ and R₇ can form, together with the nitrogen atom to which they are attached, a heterocycle selected from pyrrolidine, 3-pyrroline, optionally substituted with one or two alkyl(C₁-C₆), 1,2,3,6-tetrahydropyridine, piperidine, optionally substituted with one or two substituents independently selected from alkyl(C₁-C₆) and OH, morpholine, optionally substituted with one or two alkyl(C₁-C₆), thiomorpholine and piperazine, optionally substituted with one or two substituents independently selected from alkyl(C₁-C₆) and COalkyl(C₁-C₆).

In another preferred embodiment, R₅ is NR₆R₇ and R₆ and R₇ are independently selected from the group consisting of hydrogen, ethyl, propyl, iso-propyl, butyl, hexyl, allyl, hydroxyethyl, cyclopropylmethyl, 1,3,4-thiadiazol-2-yl, 1,3-dimethyl-5-pyrazolyl, 1-methyl-3-pyrazolyl, 2,5-dimethyl-3-pyrrolinyl, 1,2,3,6-tetrahydropiperidinyl, 2,5-dimethyl-3-pyrazolyl, 2-acetyl-3-thienyl, 2-indanyl, 2-methyl-3-pyrazolyl, 2-methyl-5-indolyl, 2-pirazinyl, 2-pyrimidinyl, 2-quinolyl, 2-thiazolyl, 3,5-isoxazol-4-yl, 3-isoxazolyl, 3-methyl-2-thienylmethyl, 3-methyl-5-isoxazolyl, 3-methyl-6-pyridyl, 3-methyl isoxazol-5-yl, 3-oxazolyl, 3-pyrazolyl, 4-methyl-2-thiazolyl, 5-trifluoromethyl-1,3,4-thiadiazol-2-yl, 5-cyclopropyl-3-pyrazolyl, 5-methyl-2-pyridyl, 5-methyl-3-pyrazolyl, 5-methyl-isoxazol-3-yl, 5-nitro-2-thiazol-2-yl, 6-methoxy-4-pyrimidinyl, 2-furylmethyl, 2-pyridylmethyl, 2-thienylethyl, phenyl, p-tolyl, 4-trifluoromethylphenyl, 4-dimethylaminophenyl, 4-fluorophenyl and 4-methoxyphenyl; or R₆ and R₇ form, together with the nitrogen atom to which they are attached, pyrrole, 2,5-dimethyl-3-pyrroline, piperidine, 1,2,3,6-tetrahydropyridine, 4-hydroxypiperidine, 3,5-dimethylpiperidine, morpholine, 2,6-dimethylmorpholine, 4-methylpiperazine and 4-acetylpiperazine.

The term “pharmaceutically acceptable salts” means salts which are acceptable for administration to a patient, such as a mammal (e.g., salts having acceptable mammalian safety for a given dosage regime). Such salts can be derived from pharmaceutically acceptable inorganic or organic bases and from pharmaceutically acceptable inorganic or organic acids. Salts derived from pharmaceutically acceptable inorganic bases include ammonium, calcium, copper, ferric, ferrous, lithium, magnesium, manganic, manganous, potassium, sodium, zinc and the like. Particularly preferred are ammonium, calcium, magnesium, potassium and sodium salts. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine; 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. Salts derived from pharmaceutically acceptable acids include acetic, ascorbic, benzenesulfonic, benzoic, camphosulfonic, citric, ethanesulfonic, edisylic, fumaric, gentisic, gluconic, glucoronic, glutamic, hippuric, hydrobromic, hydrochloric, isethionic, lactic, lactobionic, maleic, malic, mandelic, methanesulfonic, mucic, naphthalenesulfonic, naphthalene-1,5-disulfonic, naphthalene-2,6-disulfonic, nicotinic, nitric, orotic, pamoic, pantothenic, phosphoric, succinic, sulfuric, tartaric, p-toluenesulfonic, xinafoic and the like. Particularly preferred are citric, hydrobromic, hydrochloric, isethionic, maleic, naphthalene-1,5-disulfonic, phosphoric, sulfuric and tartaric acids.

Halogen or halo means F, Cl, Br, and I.

Alkyl(C₁-C₆) means a straight or branched alkyl chain with 1 to 6 carbon atoms such as methyl, ethyl, n-propyl, I-propyl, n-butyl, sec-butyl, I-butyl, t-butyl, n-pentyl and n-hexyl.

Alkenyl(C₂-C₆) means a straight or branched alkenyl chain with 2 to 6 carbon atoms such as allyl.

Specific compounds of formula (I) are selected from the group consisting of:
5-Methyl-1-(2-morpholin-4-yl-2-oxo-ethyl)-2-phenyl-1H-quinolin-4-one;
N,N-Diethyl-2-(7-methyl-4-oxo-2-phenyl-4H-quinolin-1-yl)-acetamide;
7-Methyl-1-(2-morpholin-4-yl-2-oxo-ethyl)-2-phenyl-1H-quinolin-4-one;
(7-Methyl-4-oxo-2-phenyl-4H-quinolin-1-yl)-acetic acid ethyl ester;
N,N-Dibutyl-2-(7-methyl-4-oxo-2-phenyl-4H-quinolin-1-yl)-acetamide;
N,N-Diisopropyl-2-(5-methyl-4-oxo-2-phenyl-4H-quinolin-1-yl)-acetamide;
5-Methyl-1-(2-oxo-2-pyrrolidin-1-yl-ethyl)-2-phenyl-1H-quinolin-4-one;
2-(7-Methyl-4-oxo-2-phenyl-4H-quinolin-1-yl)-N,N-dipropyl-acetamide;
N,N-Dihexyl-2-(5-methyl-4-oxo-2-phenyl-4H-quinolin-1-yl)-acetamide;
5-Methyl-1-(2-oxo-2-piperidin-1-yl-ethyl)-2-phenyl-1H-quinolin-4-one;
5-Methyl-1-(2-oxo-2-pyrrolidin-1-yl-ethyl)-2-phenyl-1H-quinolin-4-one;
7-Methyl-1-(2-oxo-2-pyrrolidin-1-yl-ethyl)-2-phenyl-1H-quinolin-4-one;
N,N-Dibutyl-2-(7-methyl-4-oxo-3-phenyl-4H-quinolin-1-yl)-acetamide;
2-(7-Methyl-4-oxo-3-phenyl-4H-quinolin-1-yl)-N,N-dipropyl-acetamide;
7-Methyl-1-(2-oxo-2-piperidin-1-yl-ethyl)-3-phenyl-1H-quinolin-4-one;
N,N-Dihexyl-2-(7-methyl-4-oxo-3-phenyl-4H-quinolin-1-yl)-acetamide;
N,N-Diethyl-2-(7-methyl-4-oxo-3-phenyl-4H-quinolin-1-yl)-acetamide;
7-Methyl-1-(2-oxo-2-pyrrolidin-1-yl-ethyl)-3-phenyl-1H-quinolin-4-one;
7-Methyl-1-(2-morpholin-4-yl-2-oxo-ethyl)-3-phenyl-1H-quinolin-4-one;
N,N-Diisopropyl-2-(7-methyl-4-oxo-3-phenyl-4H-quinolin-1-yl)-acetamide;
N,N-Dihexyl-2-(5-methyl-4-oxo-3-phenyl-4H-quinolin-1-yl)-acetamide;
5-Methyl-1-(2-oxo-2-piperidin-1-yl-ethyl)-3-phenyl-1H-quinolin-4-one;
5-Methyl-1-(2-morpholin-4-yl-2-oxo-ethyl)-3-phenyl-1H-quinolin-4-one;
N,N-Diethyl-2-(5-methyl-4-oxo-3-phenyl-4H-quinolin-1-yl)-acetamide;
N,N-Dibutyl-2-(5-methyl-4-oxo-3-phenyl-4H-quinolin-1-yl)-acetamide;
7-Methyl-1-(2-oxo-2-pyridin-3-yl-ethyl)-3-phenyl-1H-quinolin-4-one;
7-Methyl-1-(2-oxo-2-thiophen-2-yl-ethyl)-3-phenyl-1H-quinolin-4-one;
7-Methyl-1-(2-oxo-2-pyridin-2-yl-ethyl)-3-phenyl-1H-quinolin-4-one;
7-Methyl-1-(2-oxo-2-pyridin-4-yl-ethyl)-3-phenyl-1H-quinolin-4-one;
1-[2-(4-Acetyl-piperazin-1-yl)-2-oxo-ethyl]-7-methyl-3-phenyl-1H-quinolin-4-one;
7-Methyl-1-[2-(5-methyl-furan-2-yl)-2-oxo-ethyl]-3-phenyl-1H-quinolin-4-one;
1-[2-(2,6-Dimethyl-morpholin-4-yl)-2-oxo-ethyl]-7-methyl-3-phenyl-1H-quinolin-4-one;
1-[2-(2,4-Dimethyl-oxazol-5-yl)-2-oxo-ethyl]-7-methyl-3-phenyl-1H-quinolin-4-one;
7-Methyl-1-(2-oxo-2-pyrazin-2-yl-ethyl)-3-phenyl-1H-quinolin-4-one;
7-Methyl-1-[2-(4-methyl-piperazin-1-yl)-2-oxo-ethyl]-3-phenyl-1H-quinolin-4-one;
2-[3-(4-Methoxy-phenyl)-7-methyl-4-oxo-4H-quinolin-1-yl]-N,N-dipropyl-acetamide;
2-[3-(4-Chloro-phenyl)-7-methyl-4-oxo-4H-quinolin-1-yl]-N,N-dipropyl-acetamide;
2-(7-Methyl-4-oxo-3-p-tolyl-4H-quinolin-1-yl)-N,N-dipropyl-acetamide;
N-Cyclopropylmethyl-2-(7-methyl-4-oxo-3-phenyl-4H-quinolin-1-yl)-N-propyl-acetamide;
1-[2-(3,5-Dimethyl-piperidin-1-yl)-2-oxo-ethyl]-7-methyl-3-phenyl-1H-quinolin-4-one;
1-[2-(3,6-Dihydro-2H-pyridin-1-yl)-2-oxo-ethyl]-7-methyl-3-phenyl-1H-quinolin-4-one;
1-[2-(2,5-Dimethyl-2,5-dihydro-pyrrol-1-yl)-2-oxo-ethyl]-7-methyl-3-phenyl-1H-quinolin-4-one;
N,N-Diallyl-2-(7-methyl-4-oxo-3-phenyl-4H-quinolin-1-yl)-acetamide;
2-(7-Bromo-4-oxo-3-phenyl-4H-quinolin-1-yl)-N,N-dipropyl-acetamide;
2-(5-Bromo-4-oxo-3-phenyl-4H-quinolin-1-yl)-N,N-dipropyl-acetamide;
2-(4-Oxo-3,7-diphenyl-4H-quinolin-1-yl)-N,N-dipropyl-acetamide;
2-(4-Oxo-3-phenyl-4H-quinolin-1-yl)-N,N-dipropyl-acetamide;
2-(4-Oxo-3,5-diphenyl-4H-quinolin-1-yl)-N,N-dipropyl-acetamide;
(7-Methyl-4-oxo-3-p-tolyl-4H-quinolin-1-yl)-acetic acid methyl ester;
(7-Methyl-4-oxo-3-p-tolyl-4H-quinolin-1-yl)-acetic acid;
[3-(4-Chloro-phenyl)-7-methyl-4-oxo-4H-quinolin-1-yl]-acetic acid methyl ester;
[3-(4-Fluoro-phenyl)-7-methyl-4-oxo-4H-quinolin-1-yl]-acetic acid methyl ester;
[3-(4-Fluoro-phenyl)-7-methyl-4-oxo-4H-quinolin-1-yl]-acetic acid;
2-[3-(4-Chloro-phenyl)-7-methyl-4-oxo-4H-quinolin-1-yl]-N-phenyl-acetamide;
2-[3-(4-Chloro-phenyl)-7-methyl-4-oxo-4H-quinolin-1-yl]-N-pyrazin-2-yl-acetamide;
[3-(4-Chloro-phenyl)-7-methyl-4-oxo-4H-quinolin-1-yl]-acetic acid;
[3-(4-Chloro-phenyl)-7-methyl-4-oxo-4H-quinolin-1-yl]-acetic acid hydrazide;
2-(7-Methyl-4-oxo-3-p-tolyl-4H-quinolin-1-yl)-N-phenyl-acetamide;
2-[3-(4-Fluoro-phenyl)-7-methyl-4-oxo-4H-quinolin-1-yl]-N-phenyl-acetamide;
2-(7-Methyl-4-oxo-3-p-tolyl-4H-quinolin-1-yl)-N-thiazol-2-yl-acetamide;
N-(5-Methyl-isoxazol-3-yl)-2-(7-methyl-4-oxo-3-p-tolyl-4H-quinolin-1-yl)-acetamide;
[3-(4-Fluoro-phenyl)-7-methyl-4-oxo-4H-quinolin-1-yl]-acetic acid hydrazide;
(7-Methyl-4-oxo-3-p-tolyl-4H-quinolin-1-yl)-acetic acid hydrazide;
2-[3-(4-Chloro-phenyl)-7-methyl-4-oxo-4H-quinolin-1-yl]-N-(5-methyl-isoxazol-3-yl)-acetamide;
2-[3-(4-Fluoro-phenyl)-7-methyl-4-oxo-4H-quinolin-1-yl]-N-(5-methyl-isoxazol-3-yl)-acetamide;
N-Ethyl-N-(2-hydroxy-ethyl)-2-(7-methyl-4-oxo-3-phenyl-4H-quinolin-1-yl)-acetamide;
N-(2-Hydroxy-ethyl)-2-(7-methyl-4-oxo-3-phenyl-4H-quinolin-1-yl)-N-propyl-acetamide;
N,N-Diallyl-2-(5-methyl-4-oxo-3-phenyl-4H-quinolin-1-yl)-acetamide;
N-(2-Hydroxy-ethyl)-2-(5-methyl-4-oxo-3-phenyl-4H-quinolin-1-yl)-N-propyl-acetamide;
(7-Methyl-4-oxo-3-phenyl-4H-quinolin-1-yl)-acetic acid;
(5-Methyl-4-oxo-3-phenyl-4H-quinolin-1-yl)-acetic acid;
[3-(4-Chloro-phenyl)-7-methyl-4-oxo-4H-quinolin-1-yl]-acetic acid N-methyl-hydrazide;
(7-Methyl-4-oxo-3-p-tolyl-4H-quinolin-1-yl)-acetic acid N-methyl-hydrazide;
[3-(4-Fluoro-phenyl)-7-methyl-4-oxo-4H-quinolin-1-yl]-acetic acid N-methyl-hydrazide;
2-[3-(4-Fluoro-phenyl)-7-methyl-4-oxo-4H-quinolin-1-yl]-N-thiazol-2-yl-acetamide;
2-[3-(4-Chloro-phenyl)-7-methyl-4-oxo-4H-quinolin-1-yl]-N-thiazol-2-yl-acetamide;
2-[3-(4-Methoxy-phenyl)-7-methyl-4-oxo-4H-quinolin-1-yl]-N-(4-trifluoromethyl-phenyl)-acetamide;
2-(7-Methyl-4-oxo-3-phenyl-4H-quinolin-1-yl)-N-(4-trifluoromethyl-phenyl)-acetamide;
N-(4-Methoxy-phenyl)-2-(7-methyl-4-oxo-3-phenyl-4H-quinolin-1-yl)-acetamide;
N-(4-Methoxy-phenyl)-2-[3-(4-methoxy-phenyl)-7-methyl-4-oxo-4H-quinolin-1-yl]acetamide;
2-[3-(4-Methoxy-phenyl)-7-methyl-4-oxo-4H-quinolin-1-yl]-N-(5-methyl-2H-pyrazol-3-yl)-acetamide;
2-(7-Methyl-4-oxo-3-phenyl-4H-quinolin-1-yl)-N-thiazol-2-yl-acetamide;
2-[3-(4-Methoxy-phenyl)-7-methyl-4-oxo-4H-quinolin-1-yl]-N-thiazol-2-yl-acetamide;
2-(7-Methyl-4-oxo-3-phenyl-4H-quinolin-1-yl)-N-(3-methyl-thiophen-2-ylmethyl)-acetamide;
2-[3-(4-Methoxy-phenyl)-7-methyl-4-oxo-4H-quinolin-1-yl]-N-(3-methyl-isoxazol-5-yl)-acetamide;
2-[3-(4-Methoxy-phenyl)-7-methyl-4-oxo-4H-quinolin-1-yl]-N-(5-methyl-pyridin-2-yl)-acetamide;
2-(7-Methyl-4-oxo-3-phenyl-4H-quinolin-1-yl)-N-pyrimidin-2-yl-acetamide;
N-Furan-2-ylmethyl-2-(7-methyl-4-oxo-3-phenyl-4H-quinolin-1-yl)-acetamide;
N-(2,5-Dimethyl-2H-pyrazol-3-yl)-2-[3-(4-methoxy-phenyl)-7-methyl-4-oxo-4H-quinolin-1-yl]-acetamide;
N,N-Diethyl-2-[3-(4-methoxy-phenyl)-7-methyl-4-oxo-4H-quinolin-1-yl]-acetamide;
3-(4-Methoxy-phenyl)-7-methyl-1-(2-oxo-2-pyrrolidin-1-yl-ethyl)-1H-quinolin-4-one;
3-(4-Methoxy-phenyl)-7-methyl-1-(2-oxo-2-piperidin-1-yl-ethyl)-1H-quinolin-4-one;
N-Ethyl-N-(2-hydroxy-ethyl)-2-[3-(4-methoxy-phenyl)-7-methyl-4-oxo-4H-quinolin-1-yl]-acetamide;
N-(2-Hydroxy-ethyl)-2-[3-(4-methoxy-phenyl)-7-methyl-4-oxo-4H-quinolin-1-yl]-N-propyl-acetamide;
N-Cyclopropylmethyl-2-[3-(4-methoxy-phenyl)-7-methyl-4-oxo-4H-quinolin-1-yl]-N-propyl-acetamide;
N,N-Bis-(2-hydroxy-ethyl)-2-[3-(4-methoxy-phenyl)-7-methyl-4-oxo-4H-quinolin-1-yl]-acetamide;
N,N-Diallyl-2-[3-(4-methoxy-phenyl)-7-methyl-4-oxo-4H-quinolin-1-yl]-acetamide;
N-(3,5-Dimethyl-isoxazol-4-yl)-2-(7-methyl-4-oxo-3-phenyl-4H-quinolin-1-yl)-acetamide;
2-(7-Methyl-4-oxo-3-phenyl-4H-quinolin-1-yl)-N-(1-methyl-1H-pyrazol-3-yl)-acetamide;
N-(2,5-Dimethyl-2H-pyrazol-3-yl)-2-(7-methyl-4-oxo-3-phenyl-4H-quinolin-1-yl)-acetamide;
2-(7-Methyl-4-oxo-3-phenyl-4H-quinolin-1-yl)-N-(5-methyl-pyridin-2-yl)-acetamide;
2-(7-Methyl-4-oxo-3-phenyl-4H-quinolin-1-yl)-N-[1,3,4]thiadiazol-2-yl-acetamide;
N-(4-Dimethylamino-phenyl)-2-(7-methyl-4-oxo-3-phenyl-4H-quinolin-1-yl)-acetamide;
N-(3-Methyl-isoxazol-5-yl)-2-(7-methyl-4-oxo-3-phenyl-4H-quinolin-1-yl)-acetamide;
2-(7-Methyl-4-oxo-3-phenyl-4H-quinolin-1-yl)-N-p-tolyl-acetamide;
2-[3-(4-Methoxy-phenyl)-7-methyl-4-oxo-4H-quinolin-1-yl]-N-(1-methyl-1H-pyrazol-3-yl)-acetamide;
1-[2-(2,5-Dimethyl-2,5-dihydro-pyrrol-1-yl)-2-oxo-ethyl]-3-(4-methoxy-phenyl)-7-methyl-1H-quinolin-4-one;
2-(7-Methyl-4-oxo-3-phenyl-4H-quinolin-1-yl)-N-(5-methyl-2H-pyrazol-3-yl)-acetamide;
2-(7-Methyl-4-oxo-3-phenyl-4H-quinolin-1-yl)-N-pyridin-2-ylmethyl-acetamide;
N-(2-Acetyl-thiophen-3-yl)-2-(7-methyl-4-oxo-3-phenyl-4H-quinolin-1-yl)-acetamide;
N-Ethyl-2-(7-methyl-4-oxo-3-phenyl-4H-quinolin-1-yl)-N-[1,3,4]thiadiazol-2-yl-acetamide;
N-(2-Methyl-1H-indol-5-yl)-2-(7-methyl-4-oxo-3-phenyl-4H-quinolin-1-yl)-acetamide;
1-[2-(3,6-Dihydro-2H-pyridin-1-yl)-2-oxo-ethyl]-7-methyl-3-phenyl-1H-quinolin-4-one;
2-(7-Methyl-4-oxo-3-phenyl-4H-quinolin-1-yl)-N-(1H-pyrazol-3-yl)-acetamide;
N-(4-Fluoro-phenyl)-2-(7-methyl-4-oxo-3-phenyl-4H-quinolin-1-yl)-acetamide;
2-(7-Methyl-4-oxo-3-phenyl-4H-quinolin-1-yl)-N-(5-trifluoromethyl-[1,3,4]thiadiazol-2-yl)-acetamide;
2-(7-Methyl-4-oxo-3-phenyl-4H-quinolin-1-yl)-N-quinolin-2-yl-acetamide;
2-(7-Methyl-4-oxo-3-phenyl-4H-quinolin-1-yl)-N-(2-methyl-2H-pyrazol-3-yl)-acetamide;
N-(5-Cyclopropyl-2H-pyrazol-3-yl)-2-(7-methyl-4-oxo-3-phenyl-4H-quinolin-1-yl)-acetamide;
N-Isoquinolin-3-yl-2-(7-methyl-4-oxo-3-phenyl-4H-quinolin-1-yl)-acetamide;
N-Indan-2-yl-2-(7-methyl-4-oxo-3-phenyl-4H-quinolin-1-yl)-acetamide;
2-(7-Methyl-4-oxo-3-phenyl-4H-quinolin-1-yl)-N-(2-thiophen-2-yl-ethyl)-acetamide;
7-Bromo-1-[2-(3,6-dihydro-2H-pyridin-1-yl)-2-oxo-ethyl]-3-phenyl-1H-quinolin-4-one;
(7-Bromo-4-oxo-3-phenyl-4H-quinolin-1-yl)-acetic acid hydrazide;
2-(7-Bromo-4-oxo-3-phenyl-4H-quinolin-1-yl)-N,N-dibutyl-acetamide;
7-Bromo-1-(2-oxo-2-pyrrolidin-1-yl-ethyl)-3-phenyl-1H-quinolin-4-one;
7-Bromo-1-(2-oxo-2-piperidin-1-yl-ethyl)-3-phenyl-1H-quinolin-4-one;
7-Bromo-1-[2-(2,6-dimethyl-morpholin-4-yl)-2-oxo-ethyl]-3-phenyl-1H-quinolin-4-one;
N,N-Diallyl-2-(7-bromo-4-oxo-3-phenyl-4H-quinolin-1-yl)-acetamide;
7-Bromo-1-[2-(2,5-dimethyl-2,5-dihydro-pyrrol-1-yl)-2-oxo-ethyl]-3-phenyl-1H-quinolin-4-one;
2-(7-Bromo-4-oxo-3-phenyl-4H-quinolin-1-yl)-N-cyclopropylmethyl-N-propyl-acetamide;
2-(7-Methyl-4-oxo-3-phenyl-4H-quinolin-1-yl)-N-(5-nitro-thiazol-2-yl)-acetamide;
N-Isoxazol-3-yl-2-(7-methyl-4-oxo-3-phenyl-4H-quinolin-1-yl)-acetamide,
2-(7-Methyl-4-oxo-3-phenyl-4H-quinolin-1-yl)-N-(4-methyl-thiazol-2-yl)-acetamide;
7-Bromo-1-(2-oxo-2-piperidin-1-yl-ethyl)-3-phenyl-1H-quinolin-4-one;
(7-Methyl-4-oxo-3-phenyl-4H-quinolin-1-yl)-acetic acid methyl ester;
(7-Methyl-4-oxo-3-phenyl-4H-quinolin-1-yl)-acetic acid;
[3-(4-Methoxy-phenyl)-7-methyl-4-oxo-4H-quinolin-1-yl]-acetic acid methyl ester;
[3-(4-Methoxy-phenyl)-7-methyl-4-oxo-4H-quinolin-1-yl]acetic acid;
(7-Bromo-4-oxo-3-phenyl-4H-quinolin-1-yl)-acetic acid;
1-[2-(2,6-Dimethyl-morpholin-4-yl)-2-oxo-ethyl]-3-(4-methoxy-phenyl)-7-methyl-1H-quinolin-4-one;
1-[2-(3,5-Dimethyl-piperidin-1-yl)-2-oxo-ethyl]-3-(4-methoxy-phenyl)-7-methyl-1H-quinolin-4-one;
N-(6-Methoxy-pyrimidin-4-yl)-2-(7-methyl-4-oxo-3-phenyl-4H-quinolin-1-yl)-acetamide;
1-[2-(4-Hydroxy-piperidin-1-yl)-2-oxo-ethyl]-7-methyl-3-phenyl-1H-quinolin-4-one;
N-Isoxazol-3-yl-2-[3-(4-methoxy-phenyl)-7-methyl-4-oxo-4H-quinolin-1-yl]-acetamide; and
1-[2-(4-Hydroxy-piperidin-1-yl)-2-oxo-ethyl]-3-(4-methoxy-phenyl)-7-methyl-1H-quinolin-4-one.

Another embodiment of the present invention is to provide a process for preparing the compounds of formula (I).

The compounds of general structure (I) can be obtained following the synthetic strategy showed below (Scheme 1):

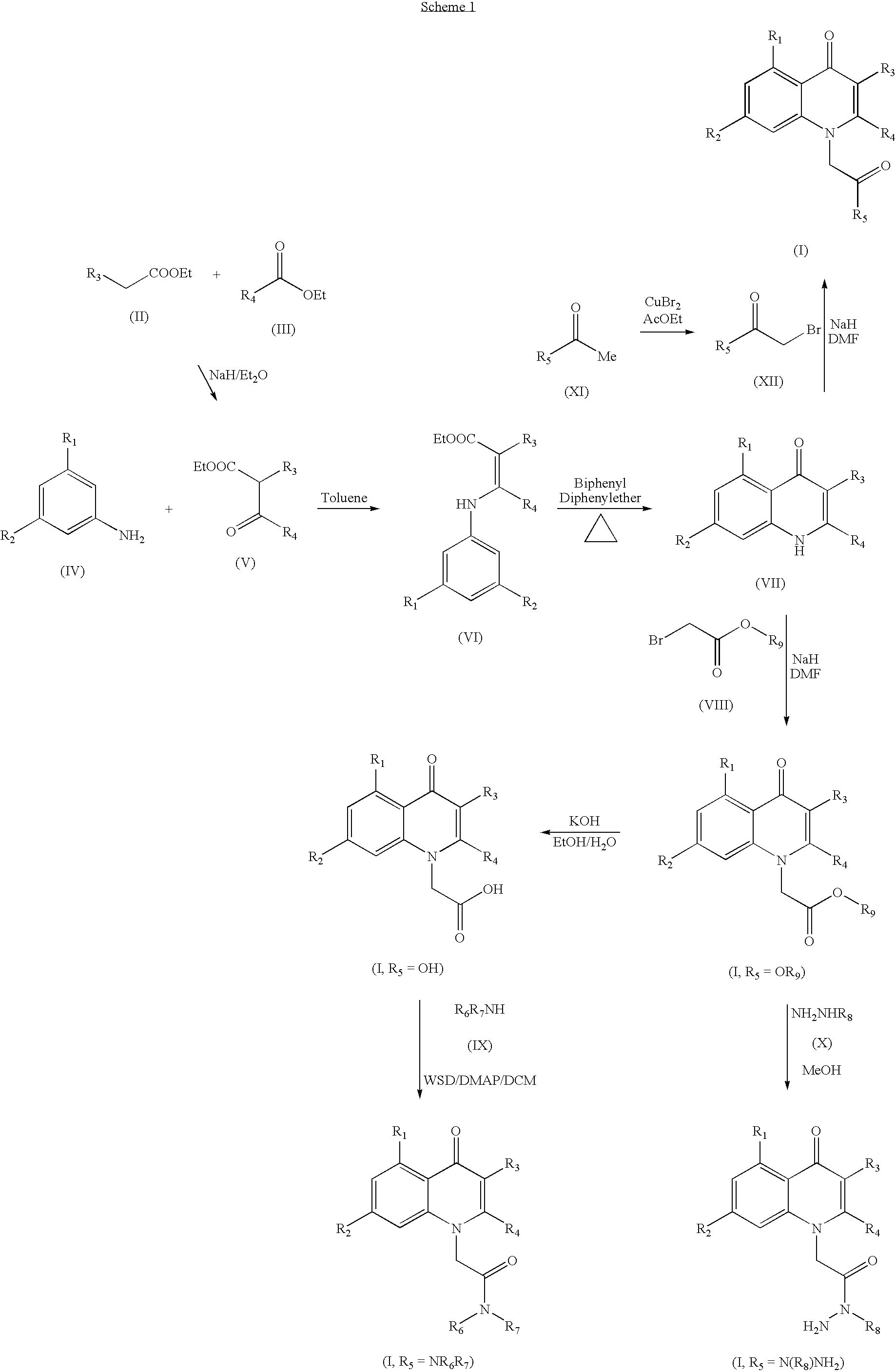

Starting from a Claisen condensation between two esters (II) and (III), it is possible to obtain the 1,3-dicarbonylic system (V), which in reaction with the aniline (IV) in toluene yields the enamine (VI). The Friedel-Crafts intramolecular condensation of (VI) at high temperature affords the quinolone system (VII). This quinolone can be converted into compounds of general structure (I) by three different ways. The first one is the N-alkylation of (VII) by using a substituted 2-bromo ethanone (XII, commercially available or synthesized from (XI) by bromination). On the other hand, the latter quinolone (VII) can be N-substituted by using an alkyl bromoacetate (VIII) to yield (I, R₅═OR₉). Methyl and ethyl bromoacetates are preferred. More preferred is methyl bromoacetate. The ester group present in (I, R₅═OR₉) can be substituted by an hydrazine (X), giving the final hydrazide compound (I, R₅═N(R₈)NH₂), Finally, esters (I, R₅═OR₉) can be converted into acids (I, R₅═OH) by conventional saponification, and these acids, by coupling with amines (IX), yield compounds (I) when the functional group to obtain is an amide.

The compounds of the present invention or their pharmaceutically acceptable salts or hydrates can be used for the preparation of a medicament for treating or preventing diseases associated with GABA_{A} receptor modulation in a human or non-human mammal. More specifically, diseases associated with GABA_{A} receptor modulation comprise diseases associated with α₁-GABA_{A} receptor modulation and/or α₂-GABA_{A} receptor modulation. A non-limitative list of such diseases comprises anxiety, epilepsy, sleep disorders, including insomnia, and the like.

Another embodiment of the present invention is to provide the use of a compound of the present invention or a pharmaceutically acceptable salt or hydrate thereof for the preparation of a medicament for treating or preventing anxiety in a human or non-human mammal.

Another embodiment of the present invention is to provide the use of a compound of the present invention or a pharmaceutically acceptable salt or hydrate thereof for the preparation of a medicament for treating or preventing epilepsy in a human or non-human mammal in need thereof.

Another embodiment of the present invention is to provide the use of a compound of the present invention or a pharmaceutically acceptable salt or hydrate thereof for the preparation of a medicament for treating or preventing sleep disorders in a human or non-human mammal in need thereof.

Another embodiment of the present invention is to provide the use of a compound of the present invention or a pharmaceutically acceptable salt or hydrate thereof for the preparation of a medicament for treating or preventing insomnia in a human or non-human mammal in need thereof.

Another embodiment of the present invention is to provide the use of a compound of the present invention or a pharmaceutically acceptable salt or hydrate thereof for the preparation of a medicament for inducing sedation-hypnosis in a human or non-human mammal in need thereof.

Another embodiment of the present invention is to provide the use of a compound of the present invention or a pharmaceutically acceptable salt or hydrate thereof for the preparation of a medicament for inducing anesthesia in a human or non-human mammal in need thereof.

Another embodiment of the present invention is to provide the use of a compound of the present invention or a pharmaceutically acceptable salt or hydrate thereof for the preparation of a medicament for modulating the necessary time to induce sleep and its duration in a human or non-human mammal in need thereof.

Another embodiment of the present invention is to provide the use of a compound of the present invention or a pharmaceutically acceptable salt or hydrate thereof for the preparation of a medicament for inducing muscle relaxation in a human or non-human mammal in need thereof.

The present invention also relates to a method of treatment or prevention of a human or non-human mammal suffering from diseases associated with GABA_{A} receptor modulation in a human or non-human mammal, which comprises administering to said human or non-human mammal in need thereof a therapeutically effective amount of a compound of the present invention or pharmaceutically acceptable salts or hydrates thereof, together with pharmaceutically acceptable diluents or carriers. More specifically, diseases associated with GABA_{A} receptor modulation comprise diseases associated with α₁-GABA_{A} receptor modulation and/or α₂-GABA_{A} receptor modulation. A non-limitative list of such diseases comprises anxiety, epilepsy, sleep disorders, including insomnia, and the like.

Further preferred embodiments of the invention relate to:

a method for treating or preventing diseases associated with the GABA_{A} receptor modulation in a human or non-human mamal in need thereof, which comprises administering to said mammal an effective amount of a compound of formula I as defined above, wherein, preferably, the GABA_{A} receptor is the α₁-GABA_{A} receptor and/or a α₂-GABA_{A} receptor;
a method for treating or preventing anxiety in a human or non-human mammal in need thereof, which comprises administering to said mammal an effective amount of a compound of formula I as defined above;
a method for treating or preventing epilepsy in a human or non-human mammal in need thereof, which comprises administering to said mammal an effective amount of a compound of formula I as defined above;
a method for treating or preventing sleep disorders in a human or non-human mammal in need thereof, which comprises administering to said mammal an effective amount of a compound of formula I as defined above;
a method for treating or preventing insomnia in a human or non-human mammal in need thereof, which comprises administering to said mammal an effective amount of a compound of formula I as defined above;
a method for inducing sedation hypnosis in a human or non-human mammal in need thereof, which comprises administering to said mammal an effective amount of a compound of formula I as defined above;
a method for inducing anesthesia in a human or non-human mammal in need thereof, which comprises administering to said mammal an effective amount of a compound of formula I as defined above;
a method for modulating the necessary time to induce sleep and its duration in a human or non-human mammal in need thereof, which comprises administering to said mammal an effective amount of a compound of formula I as defined above; and
a method for inducing muscle relaxation in a human or non-human mammal in need thereof, which comprises administering to said mammal an effective amount of a compound of formula I as defined above.

As used herein, the term “mammal” shall refer to the Mammalian class of higher vertebrates. The term “mammal” includes, but is not limited to, a human.

Another embodiment of the present invention is to provide a pharmaceutical composition containing a compound of the present invention or pharmaceutically acceptable salts and hydrates thereof, in association with therapeutically inert carriers.

The compositions include those suitable for oral, rectal and parenteral (including subcutaneous, intramuscular, and intravenous) administration, although the most suitable route will depend on the nature and severity of the condition being treated. The most preferred route of the present invention is the oral route. The compositions may be conveniently presented in unit dosage form, and prepared by any of the methods well known in the art of pharmacy.

The active compound can be combined with a pharmaceutical carrier according to conventional pharmaceutical compounding techniques. The carrier may take a wide variety of forms depending on the form of the preparation desired for administration, e.g. oral or parenteral (including intravenous injections or infusions). In preparing the compositions for oral dosage form any of the usual pharmaceutical media may be employed. Usual pharmaceutical media include, for example, water, glycols, oils, alcohols, flavoring agents, preservatives, coloring agents, and the like, in the case of oral liquid preparations (such as for example, suspensions, solutions, emulsions and elixirs); aerosols; or carriers such as starches, sugars, microcrystalline cellulose, diluents, granulating agents, lubricants, binders, disintegrating agents and the like, in the case of oral solid preparations (such as for example, powders, capsules, and tablets) with the oral solid preparations being preferred over the oral liquid preparations.

Because of their ease of administration, tablets and capsules represent the most advantageous oral dosage unit form, in which case solid pharmaceutical carriers are employed. If desired, tablets may be coated by standard aqueous or non-aqueous techniques.

A suitable dosage range for use is from about 0.01 mg to about 100.00 mg total daily dose, given as a once daily administration or in divided doses if required.

In accordance with the results obtained, certain compounds of the present invention have evidenced pharmacological activity both in vitro and in vivo, which has been similar to or higher than that of prior-art compound zolpidem. All these results support!their use in diseases or conditions modulated by α₁- and α₂-GABA_{A} receptors, such as insomnia or anesthesia, in which an induction of sleep, an induction of sedation or an induction of muscle relaxation are needed.

Thus, surprisingly, some compounds of the present invention have shown similar or higher affinity for α₁-subunit of GABA_{A} receptor, which is involved in the sedative activity. Moreover, this higher affinity correlates well with the in vivo activity in a predictive model of sedative-hypnotic activity, particularly for compounds of examples 1, 11, 14, 39, 44 and 129 (Table 3 below).

Furthermore, some compounds, in particularly compounds of examples 15, 18 and 32 of the present invention, have displayed higher α₁-/α₂-selectivity than the most selective therapeutically used compound zolpidem.

In addition, some compounds of the present invention have exhibited a higher affinity than prior art compound zolpidem for the α₂-subunit of GABA_{A} receptor, which is involved in anxiety. Compounds of examples 27 and 43 are particularly effective in anxiety related syndromes.

The pharmacological activity of the compounds of the present invention has been determined as described below.

a) Ligand-binding Assays. Determination of the Affinity of Test Compounds for α₁- and α₂-GABA_{A}, Receptor

Male Sprague-Dawley rats weighing 200-250 g at the time of experiment were used. After decapitation of the animal, the cerebellum (tissue that mostly contains α₁-GABA_{A} receptor) and spinal cord (tissue that mostly contains α₂-GABA_{A} receptor) were removed. The membranes were prepared according to the method by J. Lameh et al. (Prog. Neuro-Psychopharmacol. Biol. Psychiatry, 24, 979-991, 2000) and H. Noguchi et al. (Eur J Pharm, 434, 21-28, 2002) with slight modifications. Once the tissues weighed, they were suspended in 50 mM Tris.HCl (pH 7.4), 1:40 (v/v), or sucrose 0.32 M in the case of spinal cord, homogenized and then centrifuged at 20,000 g for 10 min at 7° C. The resulting pellet was resuspended under the same conditions and centrifuged again. The pellet was finally resuspended on a minimum volume and kept at −80° C. overnight. On the next day, the process was repeated until the final pellet was resuspended at a ratio of 1:10 (v/v) in the case of cerebellum and at a ratio of 1:5 (v/v) in the case of spinal cord.

Affinity was determined by competitive tests using radiolabeled flumazenil as ligand. The tests were performed according to the methods described by S. Arbilla et al. (Eur. J. Pharmacol., 130, 257-263, 1986); and Y. Wu et al. (Eur. J. Pharmacol., 278, 125-132, 1995) using 96-well microtiter plates. The membranes containing the study receptors, flumazenil (radiolabeling at a final concentration of 1 nM) and ascending concentrations of test compounds (in a total volume of 230 μL in 50 mM [pH 7.4] Tris.HCl buffer) were incubated. Simultaneously, the membranes were only incubated with the radiolabeled flumazenil (total binding, 100%) and in the presence of an elevated concentration of unradiolabeled flumazenil (non-specific binding, % estimation of radiolabeled ligand). The reactions started on adding the radiolabeled ligand followed by incubation for 60 minutes at 4° C. At the end of the incubation period, 200 μL of reaction were transferred to a multiscreen plate (Millipore) and filtered using a vacuum manifold and then washed three times with cold test buffer. The multiscreen plates were equipped with a GF/B filter that retained the membranes containing the receptors and the radiolabeled ligand which had been bound to the receptors. After washing, the plates were left till dry. Once dried, scintillation liquid was added and left under stirring overnight. The next day the plates were counted using a Perkin-Elmer Microbeta scintillation counter.

For analysis of the results the percentage of specific binding for every concentration of test compound was calculated as follows:

% specific binding=(X−N/T−N)×100

where,
X: amount of bound ligand for every concentration of compound.
T: total binding, maximum amount bound to the radiolabeled ligand.
N: non-specific binding, amount of radiolabeled ligand bound in a non-specific way irrespective of the receptor used.

Every concentrations of compound were tested in triplicate and their mean values were used to determine the experimental values of % specific binding versus the concentration of compound. Affinity data are expressed as % inhibition at 10⁻⁵M and 10⁻⁷M concentrations. The results of these tests are given in Tables 1 and 2.

TABLE 1
Affinity for the α₁-subunit of the GABA_{A} receptor

| 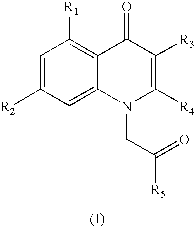 | | | | | | % inhibition | |
|---|---|---|---|---|---|---|---|
| Example | R₁ | R₂ | R₃ | R₄ | R₅ | 10⁻⁵M | 10⁻⁷M |
| 1 | Me | H | H | Ph | N-Morpholinyl | 99.5 | 57.9 |
| 11 | Me | H | H | Ph | N-Pyrrolidinyl | 99.9 | 52.6 |
| 14 | H | Me | Ph | H | N(n-Pr)₂ | 98.4 | 36.7 |
| 15 | H | Me | Ph | H | N-Piperidinyl | 87.3 | 0.0 |
| 17 | H | Me | Ph | H | NEt₂ | 91.4 | 0.7 |
| 18 | H | Me | Ph | H | N-Pyrrolidinyl | 86.2 | 7.5 |
| 27 | H | Me | Ph | H | 2-Thienyl | 97.7 | 14.0 |
| 28 | H | Me | Ph | H | 2-Pyridyl | 97.9 | 22.5 |
| 32 | H | Me | Ph | H | 2,6-Dimethyl-N-Morpholinyl | 86.2 | 18.4 |
| 33 | H | Me | Ph | H | 2,4-Dimethyl-5-Oxazolyl | 97.0 | 33.9 |
| 36 | H | Me | 4-MeOPh | H | N(n-Pr)₂ | 95.9 | 20.3 |
| 39 | H | Me | Ph | H | N(Cyclopropylmethyl)(Pr) | 98.8 | 56.3 |
| 42 | H | Me | Ph | H | 2,5-Dimethyl-3-Pyrrolinyl | 97.0 | 25.6 |
| 43 | H | Me | Ph | H | N(Allyl)₂ | 99.0 | 49.9 |
| 44 | H | Br | Ph | H | N(n-Pr)₂ | 99.8 | 61.9 |
| 47 | H | H | Ph | H | N(n-Pr)₂ | 88.1 | 2.3 |
| 80 | H | Me | 4-MeOPh | H | NH(4-MeOPh) | 78.5 | 28.5 |
| 97 | H | Me | 4-MeOPh | H | N(Allyl)₂ | 90.7 | 4.9 |
| 129 | H | Br | Ph | H | N(Allyl)₂ | 99.7 | 85.2 |

| | | |
|---|---|---|
| zolpidem | 99.8 | 51.9 |

TABLE 2
Affinity for the α₂-subunit of the GABA_{A} receptor

| 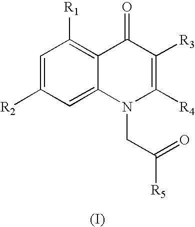 | | | | | | % inhibition | |
|---|---|---|---|---|---|---|---|
| Example | R₁ | R₂ | R₃ | R₄ | R₅ | 10⁻⁵M | 10⁻⁷M |
| 14 | H | Me | Ph | H | N(n-Pr)₂ | 77.3 | 40.6 |
| 15 | H | Me | Ph | H | N-Piperidinyl | 13.3 | 0.0 |
| 17 | H | Me | Ph | H | NEt₂ | 70.1 | 0.0 |
| 18 | H | Me | Ph | H | N-Pyrrolidinyl | 66.3 | 0.0 |
| 27 | H | Me | Ph | H | 2-Thienyl | 89.9 | 8.4 |
| 28 | H | Me | Ph | H | 2-Pyridyl | 88.0 | 0.0 |
| 32 | H | Me | Ph | H | 2,6-Dimethyl-N-Morpholinyl | 55.4 | 0.0 |
| 39 | H | Me | Ph | H | N(Cyclopropylmethyl)(Pr) | 93.4 | 28.5 |
| 42 | H | Me | Ph | H | 2,5-Dimethyl-3-Pyrrolinyl | 74.1 | 0.0 |
| 43 | H | Me | Ph | H | N(Allyl)₂ | 88.2 | 12.5 |
| 47 | H | H | Ph | H | N(n-Pr)₂ | 68.9 | 0.0 |
| 80 | H | Me | 4-MeOPh | H | NH(4-MeOPh) | 65.1 | 15.7 |
| 97 | H | Me | 4-MeOPh | H | N(Allyl)₂ | 62.0 | 0.0 |

| | | |
|---|---|---|
| zolpidem | 74.1 | 19.9 |

### b) In Vivo Determination of Predictive Sedative-Hypnotic Action

The in vivo effects of these compounds were assessed by a predictive sedation-hypnosis test in mice (D. J. Sanger et al., Eur. J. Pharmacol., 313, 35-42, 1996; and G. Griebel et al., Psychopharmacology, 146, 205-213, 1999).

Groups of 5-8 male CD1 mice, weighing 22-26 g at the time of test, were used. The test compounds were administered in single equimolecular intraperitoneal doses, suspended in 0.25% agar with one drop of Tween in a volume of 10 mL/kg. Control animals received the vehicle alone. Using a Smart System (Panlab, S. L., Spain) the traveled distance in cm was recorded for each mouse at 5 min intervals during a period of 30 minutes after dosing. The inhibition percentage traveled distance of treated animals versus control animals (the first 5 min were discarded) was calculated. The results of this test are given in Table 3.

TABLE 3
Determination of in vivo sedative-hypnotic activity in mice.

| | |
|---|---|
| | (I) |
| 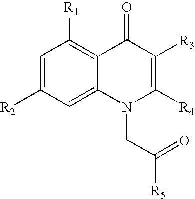 | |

| Example | R₁ | R₂ | R₃ | R₄ | R₅ | % (*) |
|---|---|---|---|---|---|---|
| 14 | H | Me | Ph | H | N(n-Pr)₂ | 96.22 |
| 15 | H | Me | Ph | H | N-Piperidinyl | 84.65 |
| 17 | H | Me | Ph | H | NEt₂ | 81.47 |
| 18 | H | Me | Ph | H | N-Pyrrolidinyl | 84.55 |
| 27 | H | Me | Ph | H | 2-Thienyl | 70.0 |
| 28 | H | Me | Ph | H | 2-Pyridyl | 84.41 |
| 32 | H | Me | Ph | H | 2,6-Dimethyl-N-Morpholinyl | 89.96 |
| 33 | H | Me | Ph | H | 2,4-Dimethyl-5-Oxazolyl | 91.67 |
| 36 | H | Me | 4-MeOPh | H | N(n-Pr)₂ | 92.06 |
| 39 | H | Me | Ph | H | N(Cyclopropylmethyl)(Pr) | 92.46 |
| 42 | H | Me | Ph | H | 2,5-Dimethyl-3-Pyrrolinyl | 92.19 |
| 43 | H | Me | Ph | H | N(Allyl)₂ | 90.99 |
| 44 | H | Br | Ph | H | N(n-Pr)₂ | 95.25 |
| 47 | H | H | Ph | H | N(n-Pr)₂ | 74.72 |
| 80 | H | Me | 4-MeOPh | H | NH(4-MeOPh) | 71.91 |
| 97 | H | Me | 4-MeOPh | H | N(Allyl)₂ | 76.76 |
| 129 | H | Br | Ph | H | N(Allyl)₂ | 93.52 |

| | |
|---|---|
| zolpidem | 91.70 |

(*) % of inhibition spontaneous motor activity (98 μmol/kg)

### Preparative Examples

### Step 1: General Method for the Claisen Condensation

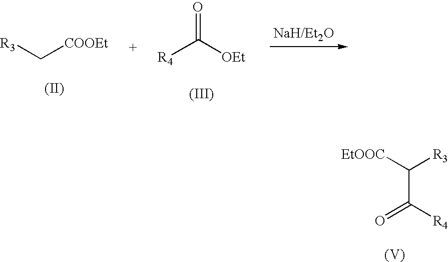

To a solution of acetate (II) (1 eq) and ester (III) (1 eq) in ethyl ether 95% sodium hydride (4 eq) was slowly added over a 2 h interval under nitrogen atmosphere. After addition was complete the solution was stirred for 2.5 h. The mixture was treated with 4.5% HCl and the organic phase was extracted with diethyl ether and dried (MgSO₄). Evaporation of the solvent gave the title compound (V) in the enolic form, which was used for the next step without further purification.

Example given for R₁═H, R₂=Me, R₃=Ph, R₄═H

¹H NMR (CDCl₃) δ 12.13 (d, J=12.6 Hz, 1H, OH), 7.31-7.25 (m, 6H, 5 Har +C═CH), 4.29 (q, J=7.2 Hz, 2H, CH₂—O), 1.29 (t, J=7.2 Hz, 3H, CH₃).

### Step 2: General Method for the Formation of the Enamine

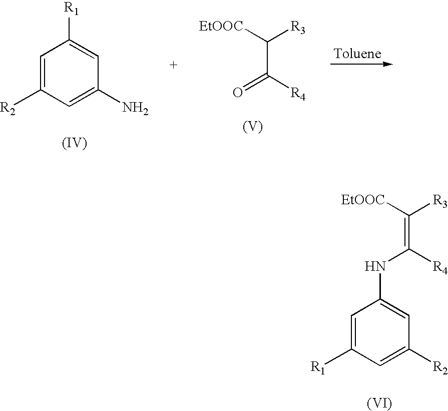

A solution of ketoester (V) (1.1 eq) and aniline (IV) (1 eq) in toluene was refluxed under stirring for 19 hours in a Dean-Stark apparatus. After cooling toluene (18 mL) was added and the mixture was treated with 10% HCl and water. The organic phase was dried with MgSO₄ and the residue obtained after evaporation was purified by silica gel column chromatography (hexane/ethyl acetate), to give the title compound (VI).

Example given for R₁═H, R₂=Me, R₃=Ph, R₄═H

¹H NMR (CDCl₃) δ 10.30 (d, J=12.6 Hz, 1H, NH), 7.43-6.83 (m, 10H, 9 Har +C═CH), 4.26 (q, J=7.2 Hz, 2H, CH₂—O), 2.33 (s, 3H, CH₃-Ph), 1.30 (t, J=7.2 Hz, 3H, CH₃).

### Step 3: General Method for the Friedel-Crafts Cyclisation

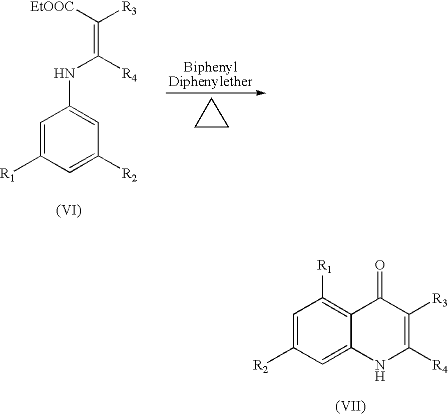

To a solution of biphenyl and diphenyl ether (1:7 molar ratio) heated at 280° C., enamine (VI) was slowly added and the reaction mixture was stirred at 280° C. for 20 min. The mixture was cooled with an ice bath and the precipitate was filtered and washed with hexane to give a mixture of the two possible isomers. These were separated by column chromatography (hexane/ethyl acetate) to give the desired product (VII).

Example given for R₁═H, R₂=Me, R₃=Ph, R₄═H

¹H NMR (DMSO-d₆) δ 11.9 (br, 1H, NH), 8.09-7.14 (m, 8H, Har), 2.44 (s, 3H, CH₃).

### Step 4: General Method for the N-alkylation (by Using (VIII) or (XII))

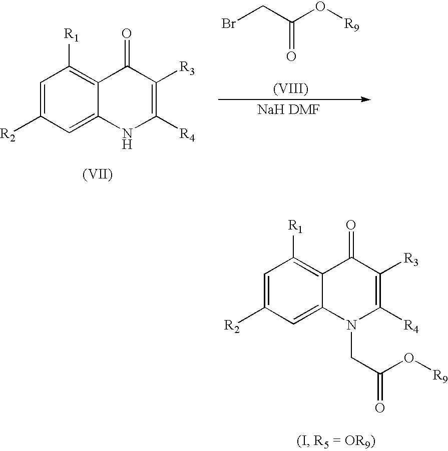

To a suspension of quinolone (VII) (1 eq) in dry dimethylformamide (DMF) under nitrogen atmosphere, 95% sodium hydride (1.6 eq) was slowly added. After addition was complete, a solution of methyl bromoacetate (VIII, R₉=Me) (2 eq) in dry DMF was added and the mixture was heated to 95° C. for 3.5 hours. After cooling, the mixture was treated with brine and extracted with ethyl acetate. The organic phase was dried (MgSO₄) and the solvent was evaporated. The residue obtained was purified by silica gel column chromatography (hexane/ethyl acetate) to give the title compound (I, R₅═OMe).

Example given for R₁═H, R₂=Me, R₃=Ph, R₄═H

¹H NMR (CDCl₃) δ 8.38 (d, J=8.1 Hz, 1H, Har), 7.63-7.60 (m, 2H, Har), 7.47 (s, 1H, CH═C), 7.36-7.12 (m, 5H, Har), 6.75 (s, 1H, Har), 4.77 (s, 2H, CH₂—C═O), 4.10 (s, 3H, OMe), 2.41 (s, 3H, CH₃-Ph).

To substitute the ester group by a hydrazine residue, to a solution of 1 eq of quinolone-ester in methanol a solution of 5 eq of the corresponding hydrazine (X) in methanol was added and the mixture was heated at reflux for 24 h. The reaction was allowed to cool and the solvent was removed. Then, acetone was added to the mixture and the solid obtained was filtered off, washed with acetone and dried, to give the title compound (I, R₅═N(R₈)NH₂) with an average yield of 90%.

### Step 5: General Method to Proceed with the Saponification

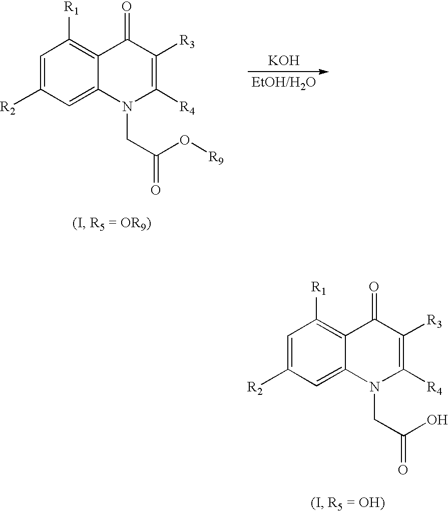

To a solution of quinolone-ester (I, R₅═OR₉) (1 eq) in ethanol an aqueous solution of KOH (3 eq) was added. The mixture was heated at reflux for 6 h. After cooling, a solution of HCl 6N was added until pH=1. The solid thus obtained was filtered off, washed with cold water and dried, to give the title compound (I, R₅═OH).

### Step 6: General Method for Amide Formation

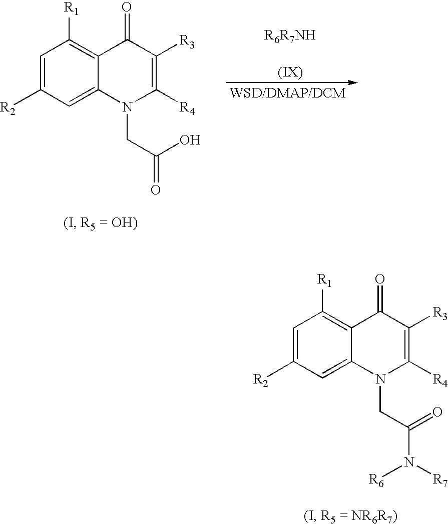

To a solution of 1 eq of the quinolone-acid in dichloromethane (DCM) a solution of 1.5 eq of water-soluble carbodiimide (WSC) in DCM was added. The mixture was stirred for 30 min at room temperature. Then, a solution of 1.5 eq of the corresponding amine and 0.5 eq of 4-(N,N-dimethylamino)-pyridine (DMAP) in DCM was added and the resulting mixture was stirred for 24 h at room temperature. The crude was extracted with HCl 1 N, and the organic layer was dried and the solvent was removed. The residue was purified by LCMS. The average yield of the process was 60%.

Example given for R₁═H, R₂=Me, R₃=Ph, R₄═H, R₆═R₇=propyl

¹H NMR (CDCl₃) δ 8.38 (d, J=8.1 Hz, 1H, Har), 7.63-7.60 (m, 2H, Har), 7.47 (s, 1H, CH═C), 7.36-7.12 (m, 4H, Har), 6.75 (s, 1H, Har), 4.77 (s, 2H, CH₂—C═O), 3.34-3.29 (m, 4H, 2 CH₂—N), 2.41 (s, 3H, CH₃-Ph), 1.78-1.51 (m, 4H, 2 CH₂—CH₃), 1.03 (t, J=7.2 Hz, 3H, CH₃), 0.87 (t, J=7.2 Hz, 3 H, CH₃);

¹³C NMR δ 175.7 (C═O), 164.9 (N—C═O), 142.6, 142.4, 139.8, 135.3, 128.5, 128.0, 127.3, 126.7, 125.1, 125.0, 121.7, 114.4 (Car+2 C═C), 53.7 (CH₂—C═O), 48.9, 48.1 (2 CH₂—N), 22.3, 22.2 (2 CH₂—CH₃), 20.8 (CH₃-Ph), 11.4, 11.4 (2 CH₃).

Compounds I-146, Table 4, were prepared according to the above reported preparative examples.

TABLE 4

| | |
|---|---|
| | (I) |
| 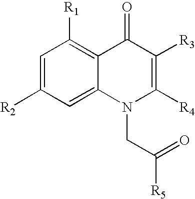 | |

| | | | | | | Purity | LCMS |
|---|---|---|---|---|---|---|---|
| Number | R₁ | R₂ | R₃ | R₄ | R₅ | (%) | (M + 1) |
| 1 | Me | H | H | Ph | N-Morpholinyl | 98 | 363.4 |
| 2 | H | Me | H | Ph | NEt₂ | 95 | 349.4 |
| 3 | H | Me | H | Ph | N-Morpholinyl | 97 | 363.4 |
| 4 | H | Me | H | Ph | NEt₂ | 97 | 322.4 |
| 5 | H | Me | H | Ph | N(n-Bu)₂ | 97 | 405.6 |
| 6 | Me | H | H | Ph | N(iPr)₂ | 99 | 377.5 |
| 7 | Me | H | H | Ph | N-Pyrrolidinyl | 97 | 347.4 |
| 8 | H | Me | H | Ph | N(n-Pr)₂ | 96 | 377.5 |
| 9 | Me | H | H | Ph | N(n-Hex)₂ | 98 | 461.7 |
| 10 | Me | H | H | Ph | N-Piperidinyl | 97 | 361.5 |
| 11 | Me | H | H | Ph | N-Pyrrolidinyl | 98 | 347.4 |
| 12 | H | Me | H | Ph | N-Pyrrolidinyl | 96 | 347.4 |
| 13 | H | Me | Ph | H | N(n-Bu)₂ | 95 | 405.6 |
| 14 | H | Me | Ph | H | N(n-Pr)₂ | 99 | 377.5 |
| 15 | H | Me | Ph | H | N-Piperidinyl | 99 | 361.5 |
| 16 | H | Me | Ph | H | N(n-Hex)₂ | 98 | 461.7 |
| 17 | H | Me | Ph | H | NEt₂ | 97 | 349.4 |
| 18 | H | Me | Ph | H | N-Pyrrolidinyl | 96 | 347.4 |
| 19 | H | Me | Ph | H | N-Morpholinyl | 100 | 363.4 |
| 20 | H | Me | Ph | H | N(iPr)₂ | 97 | 377.5 |
| 21 | Me | H | Ph | H | N(n-Hex)₂ | 98 | 461.7 |
| 22 | Me | H | Ph | H | N-Piperidinyl | 97 | 361.5 |
| 23 | Me | H | Ph | H | N-Morpholinyl | 98 | 363.4 |
| 24 | Me | H | Ph | H | NEt₂ | 97 | 349.4 |
| 25 | Me | H | Ph | H | N(n-Bu)₂ | 96 | 405.6 |
| 26 | H | Me | Ph | H | 3-Pyridyl | 100 | 355.4 |
| 27 | H | Me | Ph | H | 2-Thienyl | 98 | 360.4 |
| 28 | H | Me | Ph | H | 2-Pyridyl | 100 | 355.4 |
| 29 | H | Me | Ph | H | 4-Pyridyl | 97 | 355.4 |
| 30 | H | Me | Ph | H | N′-Acetyl-N-Piperazinyl | 99 | 404.5 |
| 31 | H | Me | Ph | H | 5-Methyl-2-Furyl | 97 | 358.4 |
| 32 | H | Me | Ph | H | 2,6-Dimethyl-N-Morpholinyl | 97 | 391.5 |
| 33 | H | Me | Ph | H | 2,4-Dimethyl-5-Oxazolyl | 95 | 373.4 |
| 34 | H | Me | Ph | H | 2-Pyrazinyl | 99 | 356.4 |
| 35 | H | Me | Ph | H | N′-Methyl-N-Piperazinyl | 96 | 376.5 |
| 36 | H | Me | 4-MeOPh | H | N(n-Pr)₂ | 97 | 407.5 |
| 37 | H | Me | 4-ClPh | H | N(n-Pr)₂ | 97 | 411.9 |
| 38 | H | Me | p-Tolyl | H | N(n-Pr)₂ | 96 | 391.5 |
| 39 | H | Me | Ph | H | N(Cyclopropylmethyl)(n-Pr) | 99 | 389.5 |
| 40 | H | Me | Ph | H | 3,5-Dimethyl-N-Piperidinyl | 98 | 389.5 |
| 41 | H | Me | Ph | H | 1,2,3,6-Tetrahydropyridin-1-yl | 98 | 359.4 |
| 42 | H | Me | Ph | H | 2,5-Dimethyl-3-Pyrrolin-1-yl | 97 | 373.5 |
| 43 | H | Me | Ph | H | N(Allyl)₂ | 96 | 373.5 |
| 44 | H | Br | Ph | H | N(n-Pr)₂ | 95 | 442.4 |
| 45 | Br | H | Ph | H | N(n-Pr)₂ | 96 | 442.4 |
| 46 | H | Ph | Ph | H | N(n-Pr)₂ | 97 | 439.6 |
| 47 | H | H | Ph | H | N(n-Pr)₂ | 95 | 363.5 |
| 48 | Ph | H | Ph | H | N(n-Pr)₂ | 97 | 439.6 |
| 49 | H | Me | p-Tolyl | H | OMe | 100 | 322.4 |
| 50 | H | Me | p-Tolyl | H | OH | 97 | 308.3 |
| 51 | H | Me | 4-ClPh | H | OMe | 98 | 342.8 |
| 52 | H | Me | 4-FPh | H | OMe | 97 | 326.3 |
| 53 | H | Me | 4-FPh | H | OH | 99 | 312.3 |
| 54 | H | Me | 4-ClPh | H | NHPh | 97 | 403.9 |
| 55 | H | Me | 4-ClPh | H | NH(2-Pyrazinyl) | 100 | 405.9 |
| 56 | H | Me | 4-ClPh | H | OH | 99 | 328.8 |
| 57 | H | Me | 4-ClPh | H | NHNH₂ | 96 | 342.8 |
| 58 | H | Me | p-Tolyl | H | NHPh | 99 | 383.5 |
| 59 | H | Me | 4-FPh | H | NHPh | 98 | 387.4 |
| 60 | H | Me | p-Tolyl | H | NH(2-Thiazolyl) | 99 | 390.5 |
| 61 | H | Me | p-Tolyl | H | NH(5-Methyl-Isoxazol-3-yl) | 96 | 388.4 |
| 62 | H | Me | 4-FPh | H | NHNH₂ | 96 | 326.3 |
| 63 | H | Me | p-Tolyl | H | NHNH₂ | 98 | 322.4 |
| 64 | H | Me | 4-ClPh | H | NH(5-Methyl-Isoxazol-3-yl) | 96 | 408.9 |
| 65 | H | Me | 4-FPh | H | NH(5-Methyl-Isoxazol-3-yl) | 96 | 392.4 |
| 66 | H | Me | Ph | H | N(Hydroxyethyl)(Et) | 100 | 365.4 |
| 67 | H | Me | Ph | H | N(Hydroxyethyl)(n-Pr) | 98 | 379.5 |
| 68 | Me | H | Ph | H | N(Allyl)₂ | 100 | 373.5 |
| 69 | Me | H | Ph | H | N(Hydroxyethyl)(n-Pr) | 98 | 379.5 |
| 70 | H | Me | Ph | H | OH | 97 | 294.3 |
| 71 | Me | H | Ph | H | OH | 96 | 294.3 |
| 72 | H | Me | 4-ClPh | H | N(Me)NH₂ | 97 | 356.8 |
| 73 | H | Me | p-Tolyl | H | N(Me)NH₂ | 97 | 336.4 |
| 74 | H | Me | 4-FPh | H | N(Me)NH₂ | 97 | 340.4 |
| 75 | H | Me | 4-FPh | H | NH(2-Thiazolyl) | 97 | 394.4 |
| 76 | H | Me | 4-ClPh | H | NH(2-Thiazolyl) | 98 | 410.9 |
| 77 | H | Me | 4-MeOPh | H | NH(4-CF₃Ph) | 98 | 467.5 |
| 78 | H | Me | Ph | H | NH(4-CF₃Ph) | 96 | 437.4 |
| 79 | H | Me | Ph | H | NH(4-MeOPh) | 97 | 399.5 |
| 80 | H | Me | 4-MeOPh | H | NH(4-MeOPh) | 96 | 429.5 |
| 81 | H | Me | 4-MeOPh | H | NH(5-Methyl-3-Pyrazolyl) | 96 | 403.5 |
| 82 | H | Me | Ph | H | NH(2-Thiazolyl) | 96 | 376.4 |
| 83 | H | Me | 4-MeOPh | H | NH(2-Thiazolyl) | 97 | 406.5 |
| 84 | H | Me | Ph | H | NH(3-Methyl-2-Thienylmethyl) | 97 | 403.5 |
| 85 | H | Me | 4-MeOPh | H | NH(3-Methyl-5-Isoxazolyl) | 96 | 404.4 |
| 86 | H | Me | 4-MeOPh | H | NH(3-Methyl-6-Pyridyl) | 98 | 414.5 |
| 87 | H | Me | Ph | H | NH(2-Pyrimidyl) | 97 | 371.4 |
| 88 | H | Me | Ph | H | NH(2-Furylmethyl) | 97 | 373.4 |
| 89 | H | Me | 4-MeOPh | H | NH(1,3-Dimethyl-5-Pyrazolyl) | 99 | 417.5 |
| 90 | H | Me | 4-MeOPh | H | NEt₂ | 99 | 379.5 |
| 91 | H | Me | 4-MeOPh | H | N-Pyrrolidinyl | 96 | 377.5 |
| 92 | H | Me | 4-MeOPh | H | N-Piperidinyl | 98 | 391.5 |
| 93 | H | Me | 4-MeOPh | H | N(Hydroxyethyl)(Et) | 96 | 395.5 |
| 94 | H | Me | 4-MeOPh | H | N(Hydroxyethyl)(n-Pr) | 96 | 409.5 |
| 95 | H | Me | 4-MeOPh | H | N(Cyclopropylmethyl)(n-Pr) | 99 | 419.5 |
| 96 | H | Me | 4-MeOPh | H | N(Hydroxyethyl)₂ | 96 | 411.5 |
| 97 | H | Me | 4-MeOPh | H | N(Allyl)₂ | 98 | 403.5 |
| 98 | H | Me | Ph | H | NH(3,5-Isoxazol-4-yl) | 99 | 388.4 |
| 99 | H | Me | Ph | H | NH(1-Methyl-3-Pyrazolyl) | 100 | 373.4 |
| 100 | H | Me | Ph | H | NH(2,5-Dimethyl-3-Pyrazolyl) | 95 | 387.5 |
| 101 | H | Me | Ph | H | NH(5-Methyl-2-Pyridinyl) | 95 | 384.5 |
| 102 | H | Me | Ph | H | NH(1,3,4-Thiadiazol-2-yl) | 98 | 377.4 |
| 103 | H | Me | Ph | H | NH(4-Dimethylamino-Ph) | 95 | 412.5 |
| 104 | H | Me | Ph | H | NH(3-Methylisoxazol-5-yl) | 98 | 374.4 |
| 105 | H | Me | Ph | H | NH(p-Tolyl) | 96 | 383.5 |
| 106 | H | Me | 4-MeOPh | H | NH(1-Methyl-3-Pyrazolyl) | 99 | 403.5 |
| 107 | H | Me | 4-MeOPh | H | NH(2,5-Dimethyl-3-Pyrrolinyl) | 96 | 403.5 |
| 108 | H | Me | Ph | H | NH(5-Methyl-3-Pyrazolyl) | 95 | 373.4 |
| 109 | H | Me | Ph | H | NH(2-Pyridylmethyl) | 98 | 384.5 |
| 110 | H | Me | Ph | H | NH(2-Acetyl-3-Thienyl) | 95 | 417.5 |
| 111 | H | Me | Ph | H | N(Et)(1,3,4-Thiadiazol-2-yl) | 99 | 405.5 |
| 112 | H | Me | Ph | H | NH(2-Methyl-5-Indolyl) | 98 | 422.5 |
| 113 | H | Me | Ph | H | NH(1,2,3,6-Tetrahydro-N-pyridyl) | 96 | 359.4 |
| 114 | H | Me | Ph | H | NH(3-Pyrazolyl) | 98 | 359.4 |
| 115 | H | Me | Ph | H | NH(4-FPh) | 97 | 387.4 |
| 116 | H | Me | Ph | H | NH[5-(CF3)-1,3,4-Thiadiazol-2-yl] | 98 | 445.4 |
| 117 | H | Me | Ph | H | NH(2-Quinolyl) | 99 | 420.5 |
| 118 | H | Me | Ph | H | NH(2-Methyl-3-Pyrazolyl) | 98 | 373.4 |
| 119 | H | Me | Ph | H | NH(5-Cyclopropyl-3-Pyrazolyl) | 98 | 399.5 |
| 120 | H | Me | Ph | H | NH(2-Quinolyl) | 97 | 420.5 |
| 121 | H | Me | Ph | H | NH(2-Indanyl) | 96 | 409.5 |
| 122 | H | Me | Ph | H | NH(2-Thienylethyl) | 99 | 403.5 |
| 123 | H | Br | Ph | H | 1,2,3,6-Tetrahydropyridyl | 97 | 424.3 |
| 124 | H | Br | Ph | H | NHNH₂ | 98 | 373.2 |
| 125 | H | Br | Ph | H | N(n-Bu)₂ | 97 | 470.4 |
| 126 | H | Br | Ph | H | N-Pyrrolidinyl | 99 | 412.3 |
| 127 | H | Br | Ph | H | N-Piperidinyl | 96 | 426.3 |
| 128 | H | Br | Ph | H | 2,6-Dimethyl-N-Morpholinyl | 97 | 456.4 |
| 129 | H | Br | Ph | H | N(Allyl)₂ | 99 | 438.3 |
| 130 | H | Br | Ph | H | 2,5-Dimethyl-3-Pyrrolinyl | 97 | 438.3 |
| 131 | H | Br | Ph | H | N(Cyclopropylmethyl)(n-Pr) | 96 | 454.4 |
| 132 | H | Me | Ph | H | NH(5-Nitro-2-Thiazol-2-yl) | 99 | 421.4 |
| 133 | H | Me | Ph | H | NH(3-Oxazolyl) | 98 | 360.4 |
| 134 | H | Me | Ph | H | NH(4-Methyl-2-Thiazolyl) | 99 | 390.5 |
| 135 | H | Br | Ph | H | N-Piperidinyl | 96 | 426.3 |
| 136 | H | Me | Ph | H | OMe | 97 | 308.3 |
| 137 | H | Me | Ph | H | OH | 98 | 294.3 |
| 138 | H | Me | 4-MeOPh | H | OMe | 96 | 338.4 |
| 139 | H | Me | 4-MeOPh | H | OH | 98 | 324.3 |
| 140 | H | Br | Ph | H | OH | 99 | 359.2 |
| 141 | H | Me | 4-MeOPh | H | 2,6-Dimethyl-N-Morpholinyl | 97 | 421.5 |
| 142 | H | Me | 4-MeOPh | H | 3,5-Dimethyl-N-Piperidinyl | 100 | 419.5 |
| 143 | H | Me | Ph | H | NH(6-Methoxy-4-Pyrimidinyl) | 96 | 401.4 |
| 144 | H | Me | Ph | H | 4-Hydroxy-N-Piperidinyl | 98 | 377.5 |
| 145 | H | Me | 4-MeOPh | H | NH(3-Isoxazolyl) | 96 | 390.4 |
| 146 | H | Me | 4-MeOPh | H | 4-Hydroxy-N-Piperidinyl | 99 | 407.5 |

### Composition Example 1

### 5 mg Tablets

| | |
|---|---|
| Active ingredient | 5.0 mg |
| Colloidal silicon dioxide | 0.6 mg |
| Croscarmellose sodium | 12.0 mg |
| Talc | 4.0 mg |
| Magnesium stearate | 1.5 mg |
| Polysorbate 80 | 1.0 mg |
| Lactose | 75.0 mg |
| Hydroxypropyl methylcellulose | 3.0 mg |
| Polyethylene glycol 4000 | 0.5 mg |
| Titanium dioxide E171 | 1.5 mg |
| Microcrystalline cellulose q.s. to | 125.0 mg |

### Composition Example 2

### 10 mg Capsules

| | |
|---|---|
| Active ingredient | 10.0 mg |
| Colloidal silicon dioxide | 0.6 mg |
| Crospovidone | 12.0 mg |
| Talc | 4.0 mg |
| Magnesium stearate | 1.5 mg |
| Lauryl sulfate sodium | 1.5 mg |
| Lactose | 77.0 mg |
| Gelatin | 28.5 mg |
| Titanium dioxide E171 | 1.5 mg |
| Indigotin E132 | 0.02 mg |
| Microcrystalline cellulose q.s. to | 155.0 mg |

### Composition Example 3

### Oral Drops

| | | |
|---|---|---|
| Active ingredient | 0.5 | g |
| Propylene glycol | 10.0 | g |
| Glycerin | 5.0 | g |
| Saccharin sodium | 0.1 | g |
| Polysorbate 80 | 1.0 | g |
| Lemon flavor | 0.2 | g |
| Ethanol | 25.0 | mL |
| Purified water q.s. to | 100.0 | mL |

### Composition Example 4

### 2.5 mg Tablets

| | |
|---|---|
| Active ingredient | 2.5 mg |
| Colloidal silicon dioxide | 0.6 mg |
| Croscaramellose sodium | 12.0 mg |
| Talc | 4.0 mg |
| Magnesium stearate | 1.5 mg |
| Polysorbate 80 | 1.0 mg |
| Lactose | 75.0 mg |
| Hydroxypropyl methylcellulose | 3.0 mg |
| Polyethylene glycol 4000 | 0.5 mg |
| Titanium dioxide E171 | 1.5 mg |
| Microcrystalline cellulose q.s. to | 125.0 mg |

### Composition Example 5

### 5 mg Capsules

| | |
|---|---|
| Active ingredient | 5.0 mg |
| Colloidal silicon dioxide | 0.6 mg |
| Crospovidone | 12.0 mg |
| Talc | 4.0 mg |
| Magnesium stearate | 1.5 mg |
| Lauryl sulfate sodium | 1.5 mg |
| Lactose | 77.0 mg |
| Gelatin | 28.5 mg |
| Titanium dioxide E171 | 1.5 mg |
| Indigotin E132 | 0.02 mg |
| Microcrystalline q.s.to | 155.0 mg |

### Composition Example 6

### Oral Drops

| | | |
|---|---|---|
| Active ingredient | 0.25 | g |
| Propylene glycol | 10.0 | g |
| Glycerin | 5.0 | g |
| Saccharin sodium | 0.1 | g |
| Polysorbate 80 | 1.0 | g |
| Lemon flavor | 0.2 | g |
| Ethanol | 25.0 | mL |
| Purified q.s. to | 100.0 | mL |

Abbreviations used throughout the specification:

Me=methyl; Et=ethyl; nPr=n-propyl; iPr=I-propyl; n-Bu=n-butyl; n-Hex=n-hexyl; Ph=phenyl

1-27. (canceled)

28. A 1H-quinolin-one compound of formula (I): 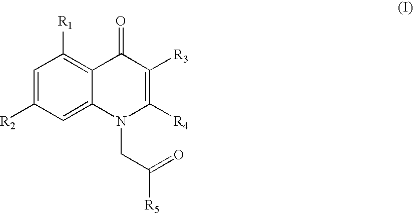
wherein
R₁ and R₂ are independently selected from the group consisting of hydrogen, alkyl(C₁-C₆), Oalkyl(C₁-C₆), halogen and phenyl;
R₃ and R₄ are independently selected from the group consisting of hydrogen and phenyl optionally substituted by alkyl(C₁-C₆), halogen and Oalkyl(C₁-C₆);
R₅ is selected from the group consisting of R₅ is selected from the group consisting of NR₆R₇, N(R₈)NH₂ and a heteroaryl ring selected from the group consisting of pyridyl, furyl, thienyl, oxazolyl, isoxazolyl, imidazolyl, pyrrolyl, pyrazolyl, pyrimidinyl and pyrazinyl, wherein each heteroaryl ring contains one or two optional alkyl(C₁-C₆) substituents;
R₆ and R₇ are independently selected from the group consisting of hydrogen; alkyl(C₁-C₆); alkenyl(C₂-C₆); cyclo alkyl (C₃-C₆)alkyl(C₁-C₆); hydroxyalkyl (C₁-C₆); hetero aryl selected from the group consisting of pyridyl, pyrimidinyl, pyrazinyl, thiazolyl, benzothiazolyl, oxazolyl, benzoxazolyl, isoxazolyl, benzisoxazolyl, pyrazolyl, furyl, benzofuryl, thienyl, benzothienyl, thiadiazolyl, pyrrolyl, imidazolyl, benzimidazolyl, indolyl, quinolyl and isoquinolyl, wherein each heteroaryl contains one or two optional substituents independently selected from the group consisting of alkyl(C₁-C₆), Oalkyl(C₁-C₆), haloalkyl(C₁-C₆), cycloalkyl(C₃-C₆), NO₂ and COalkyl(C₁-C₆); thienylalkyl(C₁-C₆), furylalkyl(C₁-C₆), pyridylalkyl (C₁-C₆); and aryl selected from the group consisting of phenyl and indanyl, wherein each aryl contains one or two optional substituents selected from the group consisting of alkyl(C₁-C₆), haloalkyl(C₁-C₆), halogen, Ndialkyl(C₁-C₆), NHalkyl(C₁-C₆), Oalkyl(C₁-C₆), NO₂, CN, OH, NH₂, COOH, COalkyl(C₁-C₆), COOalkyl(C₁-C₆), CONHalkyl(C₁-C₆), CONdialkyl(C₁-C₆) and COphenyl; with the proviso that NR₆R₇ is not NH₂, NH(C₁-C₄ alkyl) or N(C₁-C₄ alkyl)₂;
or R₆ and R₇ can form, together with the nitrogen atom to which they are attached, a heterocycle selected from pyrrolidine, 3-pyrroline, 1,2,3,6-tetrahydropyridine, piperidine, morpholine, thiomorpholine and piperazine, wherein each heterocycle contains one, two, three or four optional substituents selected from the group consisting of OH, alkyl(C₁-C₆) and COalkyl(C₁-C₆);
R₈ is selected from the group consisting of hydrogen and alkyl(C₁-C₆); and
R₉ is alkyl(C₁-C₆);
with the proviso that R₃ and R₄ cannot be hydrogen simultaneously;
and pharmaceutically acceptable salts and hydrates thereof.

29. The compound of claim 28 wherein R¹ is selected from the group consisting of hydrogen, alkyl(C₁-C₆), Oalkyl(C₁-C₆), halogen and phenyl;
R² is selected from the group consisting of hydrogen, allyl(C₁-C₆), halogen and phenyl.

30. A compound according to claim 28, wherein R₁ and R₂ are independently selected from the group consisting of hydrogen, methyl, bromine and phenyl.

31. A compound according to claim 28, wherein R₃ and R₄ are independently selected from the group consisting of hydrogen, phenyl, 4-fluorophenyl, 4-chlorophenyl, p-tolyl and 4-methoxyphenyl.

32. A compound according to claim 28, wherein R₅ is selected from the group consisting of NHNH₂, N(methyl)NH₂, 3-pyridyl, 4-pyridyl, 2-pyrazinyl, 5-methyl-2-furyl, 2-thienyl and 2,4-dimethyl-5-oxazolyl.

33. A compound according to claim 28, wherein R₆ and R₇ are independently selected from the group consisting of hexyl, allyl, hydroxyethyl, cyclopropylmethyl, 1,3,4-thiadiazol-2-yl, 1,3-dimethyl-5-pyrazolyl, 1-methyl-3-pyrazolyl, 2,5-dimethyl-3-pyrrolinyl, 1,2,3,6-tetrahydropiperidinyl, 2,5-dimethyl-3-pyrazolyl, 2-acetyl-3-thienyl, 2-indanyl, 2-methyl-3-pyrazolyl, 2-methyl-5-indolyl, 2-pyrazinyl, 2-pyrimidinyl, 2-quinolyl, 2-thiazolyl, 3,5-isoxazol-4-yl, 3-isoxazolyl, 3-methyl-2-thienylmethyl, 3-methyl-5-isoxazolyl, 3-methyl-6-pyridyl, 3-methylisoxazol-5-yl, 3-oxazolyl, 3-pyrazolyl, 1,3,4-thiadiazol-2-yl, 4-methyl-2-thiazolyl, 5-trifluoromethyl-1,3,4-thiadiazol-2-yl, 5-cyclopropyl-3-pyrazolyl, 5-methyl-2-pyridyl, 5-methyl-3-pyrazolyl, 5-methyl-isoxazol-3-yl, 5-nitro-2-thiazol-2-yl, 6-methoxy-4-pyrimidinyl, 2-furylmethyl, 2-pyridylmethyl, 2-thienylethyl, phenyl, p-tolyl, 4-trifluoromethylphenyl, 4-dimethylaminophenyl, 4-fluorophenyl and 4-methoxyphenyl; or R₆ and R₇ form, together with the nitrogen atom to which they are attached, pyrrole, 2,5-dimethyl-3-pyrroline, piperidine, 1,2,3,6-tetrahydropyridine, 4-hydroxypiperidine, 3,5-dimethylpiperidine, morpholine, 2,6-dimethylmorpholine, 4-methylpiperazine and 4-acetylpiperazine.

34. A compound selected from the group consisting of
5-Methyl-1-(2-morpholin-4-yl-2-oxo-ethyl)-2-phenyl-1H-quinolin-4-one;
N,N-Diethyl-2-(7-methyl-4-oxo-2-phenyl-4H-quinolin-1-yl)-acetamide;
7-Methyl-1-(2-morpholin-4-yl-2-oxo-ethyl)-2-phenyl-1 FI-quinolin-4-one;
(7-Methyl-4-oxo-2-phenyl-4H-quinolin-1-yl)-acetic acid ethyl ester;
N,N-Dibutyl-2-(7-methyl-4-oxo-2-phenyl-4H-quinolin-1-yl)-acetamide;
N,N-Diisopropyl-2-(5-methyl-4-oxo-2-phenyl-4H-quinolin-1-yl)-acetamide;
5-Methyl-1-(2-oxo-2-pyrrolidin-1-yl-ethyl)-2-phenyl-1H-quinolin-4-one;
2-(7-Methyl-4-oxo-2-phenyl-4H-quinolin-1-yl)-N,N-dipropyl-acetamide;
N,N-Dihexyl-2-(5-methyl-4-oxo-2-phenyl-4H-quinolin-1-yl)-acetamide;
5-Methyl-1-(2-oxo-2-piperidin-1-yl-ethyl)-2-phenyl-1H-quinolin-4-one;
5-Methyl-1-(2-oxo-2-pyrrolidin-1-yl-ethyl)-2-phenyl-1H-quinolin-4-one;
7-Methyl-1-(2-oxo-2-pyrrolidin-1-yl-ethyl)-2-phenyl-1H-quinolin-4-one;
N,N-Dibutyl-2-(7-methyl-4-oxo-3-phenyl-4H-quinolin-1-yl)-acetamide;
2-(7-Methyl-4-oxo-3-phenyl-4H-quinolin-1-yl)-N,N-dipropyl-acetamide;
7-Methyl-1-(2-oxo-2-piperidin-1-yl-ethyl)-3-phenyl-1H-quinolin-4-one;
N,N-Dihexyl-2-(7-methyl-4-oxo-3-phenyl-4H-quinolin-1-yl)-acetamide;
N,N-Diethyl-2-(7-methyl-4-oxo-3-phenyl-4H-quinolin-1-yl)-acetamide;
7-Methyl-1-(2-oxo-2-pyrrolidin-1-yl-ethyl)-3-phenyl-1H-quinolin-4-one,
7-Methyl-1-(2-morpholin-4-yl-2-oxo-ethyl)-3-phenyl-1H-quinolin-4-one,
N,N-Diisopropyl-2-(7-methyl-4-oxo-3-phenyl-4H-quinolin-1-yl)-acetamide;
N,N-Dihexyl-2-(5-methyl-4-oxo-3-phenyl-4H-quinolin-1-yl)-acetamide;
5-Methyl-1-(2-oxo-2-piperidin-1-yl-ethyl)-3-phenyl-1H-quinolin-4-one;
5-Methyl-1-(2-morpholin-4-yl-2-oxo-ethyl)-3-phenyl-1H-quinolin-4-one;
N,N-Diethyl-2-(5-methyl-4-oxo-3-phenyl-4H-quinolin-1-yl)-acetamide;
N,N-Dibutyl-2-(5-methyl-4-oxo-3-phenyl-4H-quinolin-1-yl)-acetamide;
7-Methyl-1-(2-oxo-2-pyridin-3-yl-ethyl)-3-phenyl-1H-quinolin-4-one;
7-Methyl-1-(2-oxo-2-thiophen-2-yl-ethyl)-3-phenyl-1H-quinolin-4-one;
7-Methyl-1-(2-oxo-2-pyridin-2-yl-ethyl)-3-phenyl-1H-quinolin-4-one;
7-Methyl-1-(2-oxo-2-pyridin-4-yl-ethyl)-3-phenyl-1H-quinolin-4-one;
1-[2-(4-Acetyl-piperazin-1-yl)-2-oxo-ethyl]-7-methyl-3-phenyl-1H-quinolin-4-one;
7-Methyl-1-[2-(5-methyl-furan-2-yl)-2-oxo-ethyl]-3-phenyl-1H-quinolin-4-one;
1-[2-(2,6-Dimethyl-morpholin-4-yl)-2-oxo-ethyl]-7-methyl-3-phenyl-1H-quinolin-4-one;
1-[2-(2,4-Dimethyl-oxazol-5-yl)-2-oxo-ethyl]-7-methyl-3-phenyl-1H-quinolin-4-one;
7-Methyl-1-(2-oxo-2-pyrazin-2-yl-ethyl)-3-phenyl-1H-quinolin-4-one;
7-Methyl-1-[2-(4-methyl-piperazin-1-yl)-2-oxo-ethyl]-3-phenyl-1H-quinolin-4-one;
2-[3-(4-Methoxy-phenyl)-7-methyl-4-oxo-4H-quinolin-1-yl]-N,N-dipropyl-acetamide;
2-[3-(4-Chloro-phenyl)-7-methyl-4-oxo-4H-quinolin-1-yl]-N,N-dipropyl-acetamide;
2-(7-Methyl-4-oxo-3-p-tolyl-4H-quinolin-1-yl)-N,N-dipropyl-acetamide;
N-Cyclopropylmethyl-2-(7-methyl-4-oxo-3-phenyl-4H-quinolin-1-yl)-N-propyl-acetamide;
1-[2-(3,5-Dimethyl-piperidin-1-yl)-2-oxo-ethyl]-7-methyl-3-phenyl-1H-quinolin-4-one;
1-[2-(3,6-Dihydro-2H-pyridin-1-yl)-2-oxo-ethyl]-7-methyl-3-phenyl-1H-quinolin-4-one;
1-[2-(2,5-Dimethyl-2,5-dihydro-pyrrol-1-yl)-2-oxo-ethyl]-7-methyl-3-phenyl-1H-quinolin-4-one;
N,N-Diallyl-2-(7-methyl-4-oxo-3-phenyl-4H-quinolin-1-yl)-acetamide;
2-(7-Bromo-4-oxo-3-phenyl-4H-quinolin-1-yl)-N,N-dipropyl-acetamide;
2-(5-Bromo-4-oxo-3-phenyl-4H-quinolin-1-yl)-N,N-dipropyl-acetamide;
2-(4-Oxo-3,7-diphenyl-4H-quinolin-1-yl)-N,N-dipropyl-acetamide;
2-(4-Oxo-3-phenyl-4H-quinolin-1-yl)-N,N-dipropyl-acetamide;
2-(4-Oxo-3,5-diphenyl-4H-quinolin-1-yl)-N,N-dipropyl-acetamide;
(7-Methyl-4-oxo-3-p-tolyl-4H-quinolin-1-yl)-acetic acid methyl ester;
(7-Methyl-4-oxo-3-p-tolyl-4H-quinolin-1-yl)-acetic acid;
[3-(4-Chloro-phenyl)-7-methyl-4-oxo-4H-quinolin-1-yl]-N-acetic acid methyl ester;
[3-(4-Fluoro-phenyl)-7-methyl-4-oxo-4H-quinolin-1-yl]-acetic acid methyl ester;
[3-(4-Fluoro-phenyl)-7-methyl-4-oxo-4H-quinolin-1-yl]-acetic acid;
2-[3-(4-Chloro-phenyl)-7-methyl-4-oxo-4H-quinolin-1-yl]-N-phenyl-acetamide;
2-[3-(4-Chloro-phenyl)-7-methyl-4-oxo-4H-quinolin-1-yl]-N-pyrazin-2-yl-acetamide;
[3-(4-Chloro-phenyl)-7-methyl-4-oxo-4H-quinolin-1-yl]-acetic acid;
[3-(4-Chloro-phenyl)-7-methyl-4-oxo-4H-quinolin-1-yl]-acetic acid hydrazide;
2-(7-Methyl-4-oxo-3-p-tolyl-4H-quinolin-1-yl)-N-phenyl-acetamide;
2-[3-(4-Fluoro-phenyl)-7-methyl-4-oxo-4H-quinolin-1-yl]-N-phenyl-acetamide;
2-(7-Methyl-4-oxo-3-p-tolyl-4H-quinolin-1-yl)-N-thiazol-2-yl-acetamide;
N-(5-Methyl-isoxazol-3-yl)-2-(7-methyl-4-oxo-3-p-tolyl-4H-quinolin-1-yl)-acetamide;
[3-(4-Fluoro-phenyl)-7-methyl-4-oxo-4H-quinolin-1-yl]-acetic acid hydrazide;
(7-Methyl-4-oxo-3-p-tolyl-4H-quinolin-1-yl)-acetic acid hydrazide;
2-[3-(4-Chloro-phenyl)-7-methyl-4-oxo-4H-quinolin-1-yl]-N-(5-methyl-isoxazol-3-yl)-acetamide;
2-[3-(4-Fluoro-phenyl)-7-methyl-4-oxo-4H-quinolin-1-yl]-N-(5-methyl-isoxazol-3-yl)-acetamide;
N-Ethyl-N-(2-hydroxy-ethyl)-2-(7-methyl-4-oxo-3-phenyl-4H-quinolin-1-yl)-acetamide,
N-(2-Hydroxy-ethyl)-2-(7-methyl-4-oxo-3-phenyl-4H-quinolin-1-yl)-N-propyl-acetamide;
N,N-Diallyl-2-(5-methyl-4-oxo-3-pentyl-4H-quinolin-1-yl)-acetamide;
N-(2-Hydroxy-ethyl)-2-(5-methyl-4-oxo-3-phenyl-4H-quinolin-1-yl)-N-propyl-acetamide;
(7-Methyl-4-oxo-3-phenyl-4H-quinolin-1-yl)-acetic acid;
(5-Methyl-4-oxo-3-phenyl-4H-quinolin-1-yl)-acetic acid;
[3-(4-Chloro-phenyl)-7-methyl-4-oxo-4H-quinolin-1-yl]-acetic acid N-methyl-hydrazide;
(7-Methyl-4-oxo-3-p-tolyl-4H-quinolin-1-yl)-acetic acid N-methyl-hydrazide;
[3-(4-Fluoro-phenyl)-7-methyl-4-oxo-4H-quinolin-1-yl]-acetic acid N-methyl-hydrazide;
2-[3-(4-Fluoro-phenyl)-7-methyl-4-oxo-4H-quinolin-1-yl]-N-thiazol-2-yl-acetamide;
2-[3-(4-Chloro-phenyl)-7-methyl-4-oxo-4H-quinolin-1-yl]-N-thiazol-2-yl-acetamide;
2-[3-(4-Methoxy-phenyl)-7-methyl-4-oxo-yl]-N-(4-trifluoromethyl-phenyl)-acetamide;
2-(7-Methyl-4-oxo-3-phenyl-4H-quinolin-1-yl)-N-(4-trifluoromethyl-phenyl)-acetamide;
N-(4-Methoxy-phenyl)-2-(7-methyl-4-oxo-3-phenyl-4H-quinolin-1-yl)-acetamide;
N-(4-Methoxy-phenyl)-2-[3-(4-methoxy-phenyl)-7-methyl-4-oxo-4H-quinolin-1-yl]-acetamide;
2-[3-(4-Methoxy-phenyl)-7-methyl-4-oxo-4H-quinolin-1-yl]-N-(5-methyl-2H-pyrazol-3-yl)-acetamide;
2-[7-Methyl-4-oxo-3-phenyl-4H-quinolin-1-yl]-N-thiazol-2-yl-acetamide;
2-[3-(4-Methoxy-phenyl)-7-methyl-4-oxo-4H-quinolin-1-yl]-N-thiazol-2-yl-acetamide;
2-(7-Methyl-4-oxo-3-phenyl-4H-quinolin-1-yl)-N-(3-methyl-thiophen-2-ylmethyl)-acetamide;
2-[3-(4-Methoxy-phenyl)-7-methyl-4-oxo-4H-quinolin-1-yl]-N-(3-methyl-isoxazol-5-yl)-acetamide;
2-[3-(4-Methoxy-phenyl)-7-methyl-4-oxo-4H-quinolin-1-yl]-N-(5-methyl-pyridin-2-yl)-acetamide;
2-(7-Methyl-4-oxo-3-phenyl-4H-quinolin-1-yl)-N-pyrimidin-2-yl-acetamide;
N-Furan-2-ylmethyl-2-(7-methyl-4-oxo-3-phenyl-4H-quinolin-1-yl)-acetamide;
N-(2,5-Dimethyl-2H-pyrazol-3-yl)-2-[3-(4-methoxy-phenyl)-7-methyl-4-oxo-4H-quinolin-1-yl]-acetamide;
N,N-Diethyl-2-[3-(4-methoxy-phenyl)-7-methyl-4-oxo-4H-quinolin-1-yl]-acetamide;
3-(4-Methoxy-phenyl)-7-methyl-1-(2-oxo-2-pyrrolidin-1-yl-ethyl)-1H-quinolin-4-one;
3-(4-Methoxy-phenyl)-7-methyl-1-(2-oxo-2-piperidin-1-yl-ethyl)-1H-quinolin-4-one;
N-Ethyl-N-(2-hydroxy-ethyl)-2-[3-(4-methoxy-phenyl)-7-methyl-4-oxo-4H-quinolin-1-yl]acetamide;
N-(2-Hydroxy-ethyl)-2-[3-(4-methoxy-phenyl)-7-methyl-4-oxo-4H-quinolin-1-yl]-N-propyl-acetamide;
N-Cyclopropylmethyl-2-[3-(4-methoxy-phenyl)-7-methyl-4-oxo-4H-quinolin-1-yl]-N-propyl-acetamide;
N,N-Bis-(2-hydroxy-ethyl)-2-[3-(4-methoxy-phenyl)-7-methyl-4-oxo-4H-quinolin-1-yl]-acetamide;
N,N-Diallyl-2-[3-(4-methoxy-phenyl)-7-methyl-4-oxo-4H-quinolin-1-yl]-acetamide;
N-(3,5-Dimethyl-isoxazol-4-yl)-2-(7-methyl-4-oxo-phenyl-4H-quinolin-1-yl)-acetamide;
2-(7-Methyl-4-oxo-3-phenyl-4H-quinolin-1-yl)-N-(1-methyl-1H-pyrazol-3-yl)-acetamide,
N-(2,5-Dimethyl-2H-pyrazol-3-yl)-2-(7-methyl-4-oxo-3-phenyl-4H-quinolin-1-yl)-acetamide;
2-(7-Methyl-4-oxo-3-phenyl-4H-quinolin-1-yl)-N-(5-methyl-pyridin-2-yl)-acetamide;
2-(7-Methyl-4-oxo-3-phenyl-4H-quinolin-1-yl)-N-[1,3,4]thiadiazol-2-yl-acetamide;
N-(4-Dimethylamino-phenyl)-2-(7-methyl-4-oxo-3-phenyl-4H-quinolin-1-yl)-acetamide;
N-(3-Methyl-isoxazol-5-yl)-2-(7-methyl-4-oxo-3-phenyl-4H-quinolin-1-yl)-acetamide;
2-(7-Methyl-4-oxo-3-phenyl-4H-quinolin-1-yl)-N-p-tolyl-acetamide;
2-[3-(4-Methoxy-phenyl)-7-methyl-4-oxo-4H-quinolin-1-yl]-N-(1-methyl-1H-pyrazol-3-yl)-acetamide;
1-[2-(2,5-Dimethyl-2,5-dihydro-pyrrol-1-yl)-2-oxo-ethyl]-3-(4-methoxy-phenyl)-7-methyl-1H-quinolin-4-one;
2-(7-Methyl-4-oxo-3-phenyl-4H-quinolin-1-yl)-N-(5-methyl-2H-pyrazol-3-yl)-acetamide;
2-(7-Methyl-4-oxo-3-phenyl-4H-quinolin-1-yl)-N-pyridin-2-ylmethyl-acetamide;
N-(2-Acetyl-thiophen-3-yl)-2-(7-methyl-4-oxo-3-phenyl-4H-quinolin-1-yl)-acetamide;
N-Ethyl-2-(7-methyl-4-oxo-3-phenyl-4H-quinolin-1-yl)-N-[1,3,4]thiadiazol-2-yl-acetamide;
N-(2-Methyl-1H-indol-5-yl)-2-(7-methyl-4-oxo-3-phenyl-4H-quinolin-1-yl)-acetamide;
1-[2-(3,6-Dihydro-2H-pyridin-1-yl)-2-oxo-ethyl]-7-methyl-3-phenyl-1H-quinolin-4-one;
2-(7-Methyl-4-oxo-3-phenyl-4H-quinolin-1-yl)-N-(1H-pyrazol-3-yl)-acetamide,
N-(4-Fluoro-phenyl)-2-(7-methyl-4-oxo-3-phenyl-4H-quinolin-1-yl)-acetamide;
2-(7-Methyl-4-oxo-3-phenyl-4H-quinolin-1-yl)-N-(5-trifluoromethyl-[1,3,4]thiadiazol-2-yl)-acetamide;
2-(7-Methyl-4-oxo-3-phenyl-4H-quinolin-1-yl)-N-quinolin-2-yl-acetamide;
2-(7-Methyl-4-oxo-3-phenyl-4H-quinolin-1-yl)-N-(2-methyl-2H-pyrazol-3-yl)-acetamide;
N-(5-Cyclopropyl-2H-pyrazol-3-yl)-2-(7-methyl-4-oxo-3-phenyl-4H-quinolin-1-yl)-acetamide;
N-Isoquinolin-3-yl-2-(7-methyl-4-oxo-3-phenyl-4H-quinolin-1-yl)-acetamide;
N-Indan-2-yl-2-(7-methyl-4-oxo-3-phenyl-4H-quinolin-1-yl)-acetamide;
2-(7-Methyl-4-oxo-3-phenyl-4H-quinolin-1-yl)-N-(2-thiophen-2-yl-ethyl)-acetamide;
7-Bromo-1-[2-(3,6-dihydro-2H-pyridin-1-yl)-2-oxo-ethyl]-3-phenyl-1H-quinolin-4-one;
(7-Bromo-4-oxo-3-phenyl-4H-quinolin-1-yl)-acetic acid hydrazide;
2-(7-Bromo-4-oxo-3-phenyl-4H-quinolin-1-yl)-N,N-dibutyl-acetamide;
7-Bromo-1-(2-oxo-2-pyrrolidin-1-yl-ethyl)-3-phenyl-1H-quinolin-4-one;
7-Bromo-1-(2-oxo-2-piperidin-1-yl-ethyl)-3-phenyl-1H-quinolin-4-one;
7-Bromo-1-[2-(2,6-dimethyl-morpholin-4-yl)-2-oxo-ethyl]-3-phenyl-1H-quinolin-4-one;
N,N-Diallyl-2-(7-bromo-4-oxo-3-phenyl-4H-quinolin-1-yl)-acetamide;
7-Bromo-1-[2-(2,5-dimethyl-2,5-dihydro-pyrrol-1-yl)-2-oxo-ethyl]-3-phenyl-1H-quinolin-4-one;
2-(7-Bromo-4-oxo-3-phenyl-4H-quinolin-1-yl)-N-cyclopropylmethyl-N-propyl-acetamide;
2-(7-Methyl-4-oxo-3-phenyl-4H-quinolin-1-yl)-N-(5-nitro-thiazol-2-yl)-acetamide;
N-Isoxazol-3-yl-2-(7-methyl-4-oxo-3-phenyl-4H-quinolin-1-yl)-acetamide;
2-(7-Methyl-4-oxo-3-phenyl-4H-quinolin-1-yl)-N-(4-methyl-thiazol-2-yl)-acetamide;
7-Bromo-1-(2-oxo-2-piperidin-1-yl-ethyl)-3-phenyl-1H-quinolin-4-one;
(7-Methyl-4-oxo-3-phenyl-4H-quinolin-1-yl)-acetic acid methyl ester;
(7-Methyl-4-oxo-3-phenyl-4H-quinolin-1-yl)-acetic acid;
[3-(4-Methoxy-phenyl)-7-methyl-4-oxo-4H-quinolin-1-yl]-acetic acid methyl ester;
[3-(4-Methoxy-phenyl)-7-methyl-4-oxo-4H-quinolin-1-yl]-acetic acid;
(7-Bromo-4-oxo-3-phenyl-4H-quinolin-1-yl)-acetic acid;
1-[2-(2,6-Dimethyl-morpholin-4-yl)-2-oxo-ethyl]-(4-methoxy-phenyl)-7-methyl-1H-quinolin-4-one;
1-[2-(3,5-Dimethyl-piperidin-1-yl)-2-oxo-ethyl]-3-(4-methoxy-phenyl)-7-methyl-1H-quinolin-4-one;
N-(6-Methoxy-pyrimidin-4-yl)-2-(7-methyl-4-oxo-3-phenyl-4H-quinolin-1-yl)-acetamide;
1-[2-(4-Hydroxy-piperidin-1-yl)-2-oxo-ethyl]-7-methyl-3-phenyl-1H-quinolin-4-one;
N-Isoxazol-3-yl-2-[3-(4-methoxy-phenyl)-7-methyl-4-oxo-4H-quinolin-1-yl]-acetamide; and
1-[2-(4-Hydroxy-piperidin-1-yl)-2-oxo-ethyl]-3-(4-methoxy-phenyl)-7-methyl-1H-quinolin-4-one; and
the pharmaceutically acceptable salts and hydrates thereof.

35. A process for preparing a compound of formula (I) as defined in claim 28, comprising reacting an intermediate compound of formula (VII): 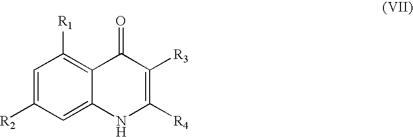
wherein R₁, R₂, R₃ and R₄ are as defined for (I), with the appropriate 2-bromo ethanone (XII):

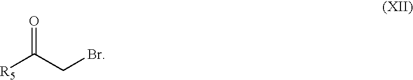

36. A process according to claim 35 for preparing an amide compound of formula (I), wherein R₅ is NR₆R₇, comprising reacting an acid compound of formula (I), wherein R₅ is OH, with the appropriate amine R₆R₇NH (IX).

37. A process according to claim 35 for preparing an hydrazide compound of formula (I), wherein R₅ is N(R₈)NH₂, comprising reacting an ester compound of formula (I), wherein R₅ is OR₉, with the appropriate hydrazine of formula NH₂NHR₈ (X).

38. Use of a compound according to claim 28 for the preparation of a medicament for treating or preventing diseases associated with the GABA_{A} receptor modulation, anxiety, epilepsy, sleep disorders or insomnia, for inducing sedation-hypnosis, anaesthesia or muscle relaxation or for modulating the necessary time to induce sleep and its duration in a human or non-human mammal in need thereof.

39. A pharmaceutical composition comprising a therapeutically effective amount of a compound as defined in claim 28, together with appropriate amounts of pharmaceutical excipients or carriers.

40. A method for treating or preventing diseases associated with the GABA_{A} receptor modulation in a human or non-human mammal in need thereof, which comprises administering to said mammal an effective amount of a compound of formula I as claimed in claim 28, or a compound of formula (I): 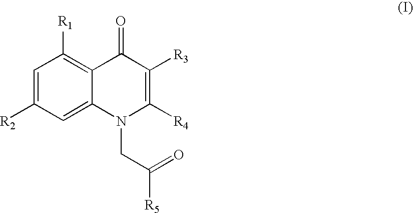
wherein
R₁ and R₂ are independently selected from the group consisting of hydrogen, alkyl(C₁-C₆), Oalkyl(C₁-C₆), halogen and phenyl;
R₃ and R₄ are independently selected from the group consisting of hydrogen and phenyl optionally substituted by alkyl(C₁-C₆), halogen and Oalkyl(C₁-C₆);
R₅ is selected from the group consisting of R₅ is selected from the group consisting of NR₆R₇, N(R₈)NH₂ and a heteroaryl ring selected from the group consisting of pyridyl, furyl, thienyl, oxazolyl, isoxazolyl, imidazolyl, pyrrolyl, pyrazolyl, pyrimidinyl and pyrazinyl, wherein each heteroaryl ring contains one or two optional alkyl(C₁-C₆) substituents;
R₆ and R₇ are independently selected from the group consisting of hydrogen; alkyl(C₁-C₆); alkenyl(C₂-C₆); cyclo alkyl (C₃-C₆)alkyl(C₁-C₆); hydroxyalkyl(C₁-C₆); hetero aryl selected from the group consisting of pyridyl, pyrimidinyl, pyrazinyl, thiazolyl, benzothiazolyl, oxazolyl, benzoxazolyl, isoxazolyl, benzisoxazolyl, pyrazolyl, furyl, benzofuryl, thienyl, benzothienyl, thiadiazolyl, pyrrolyl, imidazolyl, benzimidazolyl, indolyl, quinolyl and isoquinolyl, wherein each heteroaryl contains one or two optional substituents independently selected from the group consisting of alkyl(C₁-C₆), Oalkyl(C₁-C₆), haloalkyl(C₁-C₆), cycloalkyl(C₃-C₆), NO₂ and COalkyl(C₁-C₆); thienylalkyl(C₁-C₆), furylalkyl(C₁-C₆), pyridylalkyl(C₁-C₆); and aryl selected from the group consisting of phenyl and indanyl, wherein each aryl contains one or two optional substituents selected from the group consisting of alkyl(C₁-C₆), haloalkyl(C₁-C₆), halogen, Ndialkyl(C₁-C₆), NHalkyl(C₁-C₆), Oalkyl (C₁-C₆), NO₂, CN, OH, NH₂, COOH, COalkyl(C₁-C₆), COOalkyl(C₁-C₆), CONHalkyl(C₁-C₆), CONdialkyl(C₁-C₆) and COphenyl;
or R₆ and R₇ can form, together with the nitrogen atom to which they are attached, a heterocycle selected from pyrrolidine, 3-pyrroline, 1,2,3,6-tetrahydropyridine, piperidine, morpholine, thiomorpholine and piperazine, wherein each heterocycle contains one, two, three or four optional substituents selected from the group consisting of OH, alkyl(C₁-C₆) and COallyl(C₁-C₆);
R₈ is selected from the group consisting of hydrogen and alkyl(C₁-C₆); and
R₉ is alkyl(C₁-C₆);
with the proviso that R₃ and R₄ cannot be hydrogen simultaneously; or a compound of claim 34; or a pharmaceutically acceptable salt thereof or a hydrate thereof.

41. The method of claim 40, wherein the GABA_{A} receptor is the α₁-GABA_{A} receptor.

42. The method of claim 40 wherein the GABA_{A} receptor is the α₂-GABA_{A} receptor.

43. A method for treating or preventing anxiety in a human or non-human mammal in need thereof, which comprises administering to said mammal an effective amount of a compound of claim 40, or a pharmaceutically acceptable salt or hydrate thereof.

44. A method for treating or preventing epilepsy in a human or non-human mammal in need thereof, which comprises administering to said mammal an effective amount of a compound of claim 40, or a pharmaceutically acceptable salt or hydrate thereof.

45. A method for treating or preventing sleep disorders in a human or non-human mammal in need thereof, which comprises administering to said mammal an effective amount of a compound of claim 40, or a pharmaceutically acceptable salt or hydrate thereof.

46. A method for treating or preventing insomnia in a human or non-human mammal in need thereof, which comprises administering to said mammal an effective amount of a compound of claim 40, or a pharmaceutically acceptable salt or hydrate thereof.

47. A method for inducing sedation hypnosis in a human or non-human mammal in need thereof, which comprises administering to said mammal an effective amount of a compound of claim 40, or a pharmaceutically acceptable salt or hydrate thereof.

48. A method for inducing anesthesia in a human or non-human mammal in need thereof, which comprises administering to said mammal an effective amount of a compound of claim 40, or a pharmaceutically acceptable salt or hydrate thereof.

49. A method for modulating the necessary time to induce sleep and its duration in a human or non-human mammal in need thereof, which comprises administering to said mammal an effective amount of a compound of claim 40, or a pharmaceutically acceptable salt or hydrate thereof.

50. A method for inducing muscle relaxation in a human or non-human mammal in need thereof, which comprises administering to said mammal an effective amount of a compound of claim 40, or a pharmaceutically acceptable salt or hydrate thereof.

51. The use of a 1H-quinolin-one compound of formula (I): 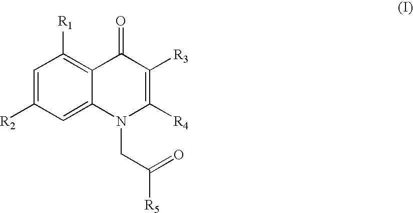
wherein
R₁ and R₂ are independently selected from the group consisting of hydrogen, alkyl(C₁-C₆), Oalkyl(C₁-C₆), halogen and phenyl;
R₃ and R₄ are independently selected from the group consisting of hydrogen and phenyl optionally substituted by alkyl(C₁-C₆), halogen and Oalkyl(C₁-C₆);
R₅ is selected from the group consisting of R₅ is selected from the group consisting of NR₆R₇, N(R₈)NH₂ and a heteroaryl ring selected from the group consisting of pyridyl, furyl, thienyl, oxazolyl, isoxazolyl, imidazolyl, pyrrolyl, pyrazolyl, pyrimidinyl and pyrazinyl, wherein each heteroaryl ring contains one or two optional alkyl(C₁-C₆) substituents;
R₆ and R₇ are independently selected from the group consisting of hydrogen; alkyl(C₁-C₆); alkenyl(C₂-C₆); cyclo alkyl (C₃-C₆) alkyl(C₁-C₆); hydroxyalkyl(C₁-C₆); hetero aryl selected from the group consisting of pyridyl, pyrimidinyl, pyrazinyl, thiazolyl, benzothiazolyl, oxazolyl, benzoxazolyl, isoxazolyl, benzisoxazolyl, pyrazolyl, furyl, benzofuryl, thienyl, benzothienyl, thiadiazolyl, pyrrolyl, imidazolyl, benzimidazolyl, indolyl, quinolyl and isoquinolyl, wherein each heteroaryl contains one or two optional substituents independently selected from the group consisting of alkyl(C₁-C₆), Oalkyl(C₁-C₆), haloalkyl(C₁-C₆), cycloalkyl(C₃-C₆), NO₂ and COalkyl(C₁-C₆); thienylalkyl(C₁-C₆), furylalkyl(C₁-C₆), pyridylalkyl(C₁-C₆); and aryl selected from the group consisting of phenyl and indanyl, wherein each aryl contains one or two optional substituents selected from the group consisting of alkyl(C₁-C₆), haloalkyl(C₁-C₆), halogen, Ndialkyl(C₁-C₆), NHalkyl(C₁-C₆), Oalkyl(C₁-C₆), NO₂, CN, OH, NH₂, COOH, COalkyl(C₁-C₆), COOalkyl(C₁-C₆), CONHalkyl(C₁-C₆), CONdialkyl(C₁-C₆) and COphenyl;
or R₆ and R₇ can form, together with the nitrogen atom to which they are attached, a heterocycle selected from pyrrolidine, 3-pyrroline, 1,2,3,6-tetrahydropyridine, piperidine, morpholine, thiomorpholine and piperazine, wherein each heterocycle contains one, two, three or four optional substituents selected from the group consisting of OH, alkyl(C₁-C₆) and COalkyl(C₁-C₆);
R₈ is selected from the group consisting of hydrogen and alkyl(C₁-C₆); and
R₉ is alkyl(C₁-C₆);
with the proviso that R₃ and R₄ cannot be hydrogen simultaneously; or of a compound of claim 34;
or a pharmaceutically acceptable salt or hydrate thereof, or
for the preparation of a medicament for treating or preventing diseases associated with the GABA_{A} receptor modulation, anxiety, epilepsy, sleep disorders or insomnia, for inducing sedation-hypnosis, anaesthesia or muscle relaxation or for modulating the necessary time to induce sleep and its duration in a human or non-human mammal in need thereof.

52. A 1H-quinoline-one compound as defined in claim 51 for use in the treatment or prevention of diseases associated with the GABA_{A} receptor modulation, anxiety, epilepsy, sleep disorders or insomnia, for inducing sedation-hypnosis, anaesthesia or muscle relaxation or for modulating the necessary time to induce sleep and its duration in a human or non-human mammal in need thereof.

53. The 1H-quinoline-one compound of claim 52, wherein the GABA_{A} receptor is the α₁-GABA_{A} receptor or the α₂-GABA_{A} receptor.
